(19) United States
(12) Patent Application Publication
(10) Pub. No.: US 2005/0176089 A1
(43) Pub. Date: Aug. 11, 2005

(54) Methods and devices for retrieving a subject's physiological history from a biological sample analysis

(76) Inventor: Michael Scott Ehrlich, Andover, MA (US)

Correspondence Address: Patrick R.H. Waller, Ph.D.;Wolf, Greenfield & Sacks, P.C., 600 Atlantic Avenue, Boston, MA, 02210, US

(21) Appl. No.: 10/774,681

(22) Filed: Feb. 8, 2004

Related U.S. Application Data
(60) Provisional application No. 60/527,205, filed on Dec. 4, 2003.

(51) Int. Cl. G06F 19/00; G01N 33/48; G01N 33/50; G01N 1/30

(52) U.S. Cl. 435/40.5; 702/21

(57) ABSTRACT

Methods and devices for retrieving a subject's physiological history from a single sample or measurement are disclosed. Methods and devices for obtaining a blood glucose history from a single blood sample are disclosed.

### RELATED APPLICATION

This application claims benefit under 35 U.S.C. 119(e) of U.S. provisional patent application 60/527,205, filed Dec. 4, 2003, the entire content of which is incorporated herein by reference.

### BACKGROUND OF INVENTION

1. Field of Invention

The invention generally relates to methods and devices for evaluating a subject's physiological history based on an analysis of data obtained from a biological sample or from a medical test. In particular, the invention relates to methods and devices for evaluating a subject's blood glucose history based on blood sample analysis.

2. Discussion of Related Art

There is an ongoing need for analytical technology to improve the medical evaluation of a subject's physiological history based on data obtained from a doctor's visit or a laboratory assay. Typical medical or laboratory tests provide a snapshot of a subject's health status at the time of the test. Some tests also provide information about the average health status of the subject over a period of time prior to the test. However, such tests fail to provide useful information about fluctuations in the subject's physiological status during the period of time prior to the test.

The analysis of blood glucose levels in patient samples illustrates the limitations of current analytical technology. Blood glucose levels can be detected directly using a number of different tests. The resulting information can be useful for real-time monitoring of blood glucose levels, for example, for a diabetic individual. However, the results provide information only about the amount of glucose in the subject's blood at the time of the test.

Another type of test that is used to detect blood glucose levels involves measuring the amount of glycated hemoglobin in a subject's blood sample. An HbA1c test, for example, measures the amount of a specific type of glycated hemoglobin. The results from this type of test do not provide information about the subject's blood glucose level at the time of the test. Instead, the amount of glycated hemoglobin provides an estimate of the subject's average blood glucose level over a period of several weeks prior to the test. However, this estimate is often inaccurate, because the total amount of glycated hemoglobin in a blood sample at the time of a test is a function not only of the level of glucose in the blood but also of the timing and duration of any change in blood glucose levels during the weeks prior to the test.

In practice, a measurement of the total amount of glycated hemoglobin in a blood sample is used to provide an estimate of a subject's blood glucose level over a period of 4-6 weeks prior to a test. However, such measurements typically don't reflect minor blood glucose level changes. In addition, such measurements don't provide any information about daily or weekly blood glucose level fluctuations. This lack of information can lead to failures in diagnosis, prognosis, and treatment.

Similarly, diagnostic methods for other diseases and conditions do not provide detailed and historical information about a subject's physiological status based on an analysis at a single time point.

Therefore, despite the existence of many medical and laboratory tests, there is still a pressing need in the art for methods and devices for obtaining more detailed information about a subject's physiological history based on an analysis of a medical test or a patient sample at a given time.

### SUMMARY OF INVENTION

The invention provides methods and devices for recovering a subject's physiological history from data obtained during a single medical or laboratory test.

The invention is useful for obtaining information about a subject's physiological history prior to the time of a test. The invention is also useful for monitoring a subject's physiological status over a pre-selected time period without requiring multiple measurements or sample withdrawals during that time period. The information provided by the invention can be used for diagnostic, prognostic, and therapeutic purposes. The invention is particularly useful for analyzing a subject's blood glucose level history, and for diagnostic, prognostic, and therapeutic purposes related to diabetes.

The invention is a process for recovering from a measurement of the current state of a system, one or more components of the state of that system at more than one time point prior to the time of the measurement.

In general, the invention involves measuring the amount of a cellular modification for each of a plurality of cells in a biological sample, and processing the measurements to obtain a profile of physiological changes over a time period prior to the time of the measurement.

In one aspect, the invention includes methods for obtaining a subject's physiological history from an analysis of the amounts of cellular modification of individual cells in a sample.

In one embodiment, the invention involves obtaining a cell distribution profile (CDP) representing the distribution of cells as a function of a measured amount of cellular modification for each cell. A CDP provides the amount of cells (absolute number of cells or relative numbers of cells) corresponding to each of a plurality of ranges of measurements. In some embodiments, a CDP can be the data output of an assay or device if the data output provides amounts of cells for different ranges of measurements. Alternatively, a CDP can be generated by processing experimental data to obtain amounts of cells corresponding to different ranges of measurements.

In another embodiment, the invention includes analytical methods for obtaining a subject's physiological history based on a CDP or other sorted or ordered data.

According to one aspect of the invention, a method of obtaining a subject's physiological history from a sample analysis is provided. The method comprises the steps of:
(a) measuring an amount of a cellular modification for each of a plurality of cells from a biological sample;
(b) sorting the cell measurements from step (a) as a function of the amount of modification; and,
(c) determining a physiological history for the subject by analyzing the ordered cell measurements.

In some preferred embodiments, the physiological history is indicative of a disease or disorder in the subject. In some embodiments, the disease or disorder is diabetes. The diabetes is Type I diabetes, Type 11 diabetes, or gestational diabetes.

In some embodiments, the disease or disorder is a thyroid disease or disorder.

Examples of thyroid diseases or disorders include: hypothyroidism, hyperthyroidism, euthyroid sick syndrome, Graves disease, juvenile hypothyroidism, and thyroid cancer.

In some embodiments, the biological sample is a blood sample.

In some embodiments, the cellular modification is hemoglobin glycation.

In some embodiments, the physiological history is determined by comparing the cell distribution profile or a portion thereof with one or more reference distribution profiles.

In some embodiments, the physiological history is determined using an algorithm comprising the steps of:
(i) calculating a change in the amount of modification for each of a series of time intervals represented by the cell distribution profile or a portion thereof, and,
(ii) determining the physiological history of the subject during the series of time intervals based on the calculated change in the amount of modification.

In some embodiments, the method further comprises the step of using the physiological history to provide a diagnosis for the subject.

In some embodiments, the method further comprises the step of using the physiological history to provide a prognosis for the subject.

In some embodiments, each of the plurality of cells is isolated before the amount of cellular modification is measured for that cell. In other embodiments, each of the plurality of cells is isolated based on the amount of cellular modification in that cell.

In some embodiments, the cell distribution profile is obtained using a procedure selected from the group consisting of chromatography, electrophoresis, mass spectrometry, and cell sorting.

According to another aspect of the invention, a method of obtaining a cell distribution profile of a subject is provided. The method comprises the steps of:
(a) obtaining a biological sample comprising intact cells;
(b) isolating a plurality of intact cells from the biological sample;
(c) measuring an amount of a physiological variable in each of the isolated cells; and,
(d) determining a cell distribution profile based on the measured amounts of the variable in the isolated cells.

According to yet another aspect of the invention, a method of obtaining a cell distribution profile of a subject is provided. The method comprises the steps of:
(a) obtaining a biological sample comprising intact cells; and
(b) determining a cell distribution profile for a plurality of the intact cells using a procedure that sorts the intact cells based on an amount of a physiological variable in each of the sorted cells.

In some embodiments, the cell distribution profile is a continuous profile. In other embodiments, the cell distribution profile is a discrete profile.

In some embodiments, the physiological variable is the amount of glycated hemoglobin in each cell.

In some embodiments, the plurality of intact cells are separated using a procedure selected from the group consisting of chromatography, electrophoresis, mass spectrometry, and cell sorting.

In some preferred embodiments the biological sample is a blood sample.

In some embodiments, a subject is a patient, preferably a human patient, diagnosed with diabetes or with abnormal blood glucose levels.

According to yet another aspect of the invention, a method for recovering the blood glucose level history of a patient diagnosed with diabetes is provided. The method comprises the steps of:
(a) obtaining a blood sample comprising intact cells
(b) obtaining a cell distribution profile by sorting a plurality of the intact cells based on the amount of glycated hemoglobin in each cell; and,
(c) determining the patient's glucose level history based on the cell distribution profile.

In some embodiments, a subject's blood glucose level history is obtained by comparing the subject's cell distribution profile to one or more reference profiles.

In other embodiments, the subject's blood glucose level history is obtained using an algorithm. In some embodiments, the algorithm corrects for non-linearity in the rate of hemoglobin glycation. In some preferred embodiments, the algorithm uses patient specific input data. Examples of patient specific input data include the patient's age, gender, and weight.

In some embodiments, the algorithm uses a value for the average life span of a red blood cell.

In some embodiments, the intact cells are sorted using a procedure selected from the group consisting of chromatography, electrophoresis, mass spectrometry, and cell sorting.

In some embodiments, the method comprises the additional step of determining whether the patient has Type I, Type II, or gestational diabetes.

In some embodiments, the method comprises the additional step of determining a treatment regimen for the patient.

In some embodiments, the method comprises the step of obtaining a second blood sample at a second time and performing steps (b) and (c) on the second blood sample.

In some embodiments, three or more blood samples are analyzed for a subject.

In some embodiments, the glycated hemoglobin is labeled.

In some embodiments, the glycated hemoglobin is labeled using a labeled antibody that is specific for glycated hemoglobin. In other embodiments, a first unlabeled antibody specific for glycated hemoblobin is used to bind the glycated hemoblobin and a second labeled antibody specific for one or more epitopes on the first antibody is used to bind to the first antibody. In further embodiments, the non-glycated hemoglobin is labeled, for example using an antibody specific for non-glycated hemoglobin. As discussed above, this antibody can be labeled directly or using a second antibody that itself is labeled. In yet further embodiments, both the glycated and non-glycated hemoglobin are labeled, preferably with different labels. Preferred labels are fluorescent labels. However, other labels can be used, including radioactive, enzymatic, and molecular weight tags.

In still another aspect of the invention, a chromatography device for separating blood cells based on levels of glycosylated hemoglobin is provided. The device comprises:
(a) a high pressure column comprising a sample chamber and a tapered exit hole;
(b) a dynamic light scattering detector connected to the exit hole; and,
(c) a gas powered injector connected to the sample chamber.

In still yet another aspect of the invention, an electrophoretic device for separating blood cells based on levels of glycated hemoglobin is provided. The device comprises:
(a) a liquid flow column with a sample entry point and a plurality of collection points along the length of the column; and,
(b) an electromagnetic field generator associated with the column, wherein the electromagnetic field is normal to the direction of the liquid flow.

In a further embodiment, a computer-readable medium is provided that stores computer-readable signals defining instructions that, as a result of being executed by a computer, instructs the computer to perform methods of the invention described in the preceding paragraphs.

In another aspect, the invention includes devices and methods for separating cells. In preferred embodiments, the cells are separated as a function of the amount of a cellular modification of each cell.

In another aspect, the invention involves diagnostic, prognostic, and therapeutic recommendations.

In another aspect, the invention provides kits including one or more solutions for stabilizing cells in a biological sample for subsequent analysis.

In another aspect, the invention provides diagnostic services.

In another aspect, the invention provides computer programs for analyzing cellular modification measurements.

In another aspect, the invention provides methods and related computer programs for analyzing data obtained from a non-invasive medical test such as an MRI, CAT scan, CT scan, ultrasonogram, Positron Emission Tomogram (PET) scan, or a Single Photon Emission Computed Tomogram (SPET).

In some embodiments, analytical or diagnostic methods of the invention are available on a web-site. The web-site can also include tables of reference information related to the invention. Algorithms of the invention can be down-loaded from a web-site. Alternatively, an operator can submit data to a web-site for analysis. The data is then processed and a profile is returned to the operator.

In some embodiments, computer programs of the invention are stored on a computer-readable medium such as a disk.

The invention can be used to detect hyperglycemia, hypoglycemia, and other defects in blood glucose regulation.

In one embodiment, the amount of glycated hemoglobin is measured for each of a plurality of red blood cells in a blood sample. A blood glucose history as a function of time can be recovered by analyzing the amount of glycated hemoglobin associated with the red blood cells in the sample. From a small sample of blood, an instantaneous blood glucose level estimate can be returned for all moments in time going back to approximately 120 days before the sample was taken. In contrast, the currently practiced HbA1c test estimates a subject's average blood glucose level over the past few months assuming that the subject's average glucose level remained relatively constant during that time interval.

### BRIEF DESCRIPTION OF DRAWINGS

The accompanying drawings, are not intended to be drawn to scale. For purposes of clarity, not every component may be labeled in every drawing. In the drawings:

FIG. 1 shows a general outline of a method of the invention;

FIGS. 2A-C show examples of a cell distributions as a function of hemoglobin glycation for subjects with different blood glucose levels;

FIG. 3 shows an example of a cell distribution as a function of hemoglobin glycation for a subject with elevated blood glucose;

FIG. 4 shows an embodiment of step 140;

FIG. 5 shows a division of a CDP into fractions each containing equal numbers of cells;

FIG. 6 shows another embodiment of step 140;

FIG. 7 shows another embodiment of step 140;

FIG. 8 shows a method for retrieving blood glucose levels as a function of sample point time (tₛₚ) when T is given;

FIG. 9 shows a method for retrieving blood glucose levels as a function of sample point time (tₛₚ) when δ is given;

FIG. 10 shows a method for retrieving blood glucose levels as a function of sample point time: FIG. 10A shows data indicating the amount of cells (Kₐ₁ through Kₐₘ) as a function of glycation intervals (δ₁ through δₘ), and in FIG. 10B the equations provide an embodiment for finding tₛₚ and g(tₛₚ) for all sample points (sp) when δ₁₋ₘ and corresponding Kₐ₁₋ₘ are given;

FIG. 11 shows an embodiment of a column for separating cells;

FIG. 12 shows an embodiment of a method for manufacturing a column for separating cells: FIG. 12A shows a side view of a column manufacturing embodiment, FIG. 12B shows a front view of a column manufacturing embodiment, and FIG. 12C shows a cross-section of a column embodiment with a coolant sheath surrounding the column;

FIG. 13 shows an embodiment of an electrophoretic device of the invention;

FIG. 14 shows an example of a subject's glucose fluctuation over the period of a day (day 112);

FIG. 15 shows an example of a cell distribution profile corresponding to a period of one day (day 112); and,

FIG. 16 shows an example of a reconstructed glucose profile for one day (day 112) based on an analysis of a cell distribution profile.

### DETAILED DESCRIPTION

The invention provides methods and devices for obtaining a profile of recent fluctuations in a subject's physiological status based on an analysis of a single biological sample. The invention is related, in part, to the realization that the amount of one or more cellular modifications of any cell in a sample reflects the cumulative historical exposure of that cell to specific cell-modifying physiological conditions. According to the invention, cells of different ages may have different amounts of cellular modification, because each cell is exposed to a different portion of a subject's physiological history. A cell sample containing cells of different age contains cells that have been exposed to overlapping portions of a subject's physiological history. The invention provides methods for analyzing a plurality of cells of different ages in order to extract the physiological history of the subject. The invention generally involves measuring an amount of a cellular modification for each of a plurality of cells in a sample, and analyzing the distribution of these cells with respect to the measured amount of cellular modification for each cell.

The invention provides more than a simple average measure of the subject's recent physiological status. The invention provides details about a subject's recent physiological changes as a function of time, based on the information obtained from a single biological sample. For example, the invention can provide a profile of daily blood-glucose level fluctuations over a 120 day period based on the information contained in a single blood sample.

The invention is useful for recovering information about past physiological fluctuations in a subject based on an analysis of a single biological sample. The invention is useful for medical diagnostic, prognostic, and therapeutic applications. The invention is also useful for evaluating drug side effects and for testing candidate drugs for disease treatment. The invention is particularly useful for diabetes diagnostics, prognostics, and treatment. The invention is also useful for other diseases and disorders as disclosed herein.

The invention generally involves analyzing the amount of a specific cellular modification of individual cells obtained from a population of cells that have a known or quantifiable turnover rate. The distribution of these cells as a function of the amount of cellular modification of each cell is then interpreted to generate a profile of recent physiological fluctuations related to the cellular modification that is being analyzed.

A useful cellular modification generally has the following properties: a) the total amount of the cellular modification of an individual cell increases with time (i.e. with the age of the cell); and b) the rate of cellular modification at any time is a function of the physiological status of the subject at that time. For example, hemoglobin glycation can be used to analyze blood-glucose fluctuations in a subject, because a) the amount of hemoglobin glycation in a red-blood cell increases with time; and b) the rate of hemoglobin glycation increases if the level of blood-glucose increases.

According to the invention, the total amount of a cellular modification of a single cell at any given time is a function of both the age of that cell and the physiological status of the subject during the life of that cell up to that point in time. Therefore, the total amount of a cellular modification in a biological sample is the sum of all the individual cellular modification amounts, and is a function of the age of each cell in the sample and the physiological status of the subject during the life of each cell up to the time of measurement. However, the physiological history of the subject cannot be obtained directly from the total amount of cellular modification in the sample. Rather, individual cells (or small groups of cells) need to be measured and analyzed in order to retrieve the subject's physiological profile.

For example, the total amount of glycated hemoglobin in a subject's blood sample is a reflection of both the age of the red blood cells in the sample and the blood-glucose profile of the subject over the life of these cells. However, the subject's physiological profile cannot be obtained from the total amount of glycated hemoglobin in the blood sample. Rather, the profile is obtained by measuring the amount of glycated hemoglobin for each of a plurality of red blood cells in the sample.

The analysis of glycated hemoglobin is based on the observation that the rate of hemoglobin glycation in red blood cells is a function of the amount of glucose in the blood. When red blood cells are created, they contain non-glycated hemoglobin. As the red blood cells mature and age, their hemoglobin is progressively glycated by reacting with blood glucose. The amount of blood glucose affects the rate of hemoglobin glycation. Increased glucose levels result in an increased accumulation of glycated hemoglobin in red blood cells. However, the total amount of glycated hemoglobin measured in a blood sample is a function of the timing of the measurement relative to the timing of any changes in blood glucose levels. Red blood cells are generated relatively constantly over the life of a subject. Red blood cells typically have an average lifespan of 120 days, after which they are removed from the blood stream. Therefore, any changes in blood glucose levels that occurred more than 120 days prior to a measurement will not be reflected in the amount of glycated hemoglobin in a subject's blood at the time of the measurement. Even glucose level changes that occurred close to 120 days prior to a test will have very little effect on the total amount of glycated hemoglobin, because the blood sample will contain only a few remaining cells from around 120 days prior to the measurement.

The total amount of hemoglobin that is measured in a typical blood-glucose assay such as an HbA1c test can be approximated mathematically as follows (neglecting non-linear effects):

$${\int }_{c = - L}^{c = 0} {\int }_{t = c}^{t = 0} g ( t ) ⅆ t ⅆ c \qquad Equation ( 1 )$$

Where
   “L” is the average lifetime of a red blood cell (typically 120 days);
   “c” is the time at which a particular red blood cell was created;
   “t” is time;
   “g(t)” is the blood glucose level as a function of time; and,
   Time “t=0” is the time at which the HbA1c test was administered.

According to the invention, the HbA1c test result can be represented by the value of the double integral of Equation (1), because the subject's red blood cells (RBCs) perform the inner integral, and the laboratory equipment performs the outer integral over the cell sample. The HbA1c test separates unbound hemoglobin from glucose-bound hemoglobin and measures the total amount of glucose-bound hemoglobin in a sample. The HbA1c result provides an estimate of average recent blood glucose levels in a subject. However, the HbA1c test does not provide information about blood glucose level fluctuations.

In contrast, methods of the invention retrieve information about blood glucose fluctuations by sorting RBCs (preferably intact RBCs) based on the amount of their hemoglobin bound to glucose. The distribution of these RBCs as a function of the amount of glucose-bound hemoglobin in each cell is then analyzed to retrieve a profile of recent blood glucose fluctuations in the subject.

As discussed above, the physiological history of each individual cell is reflected in the amount of certain cellular modifications. Cells created at different times have different amounts of cellular modification, and the physiological history of the subject can be recovered from an analysis of the amount of cellular modification of each of a plurality of individual cells generated at different times. However, the physiological history of the subject can only be recovered for times that are represented by cells in the sample.

Accordingly, a physiological profile typically can be obtained for a period of time that extends back only as far as the lifespan of the cell-type being analyzed. For example, a red blood cell has a lifespan of approximately 120 days in a human male and approximately 110 days in a human female. A single blood sample can be analyzed to generate a blood-glucose level profile that spans the 120 or 110 day period immediately prior to the time at which the blood sample was obtained. However, the blood sample does not contain information about blood glucose fluctuations that occurred more than 120 days before the date of the blood sample, because no RBCs remain from that time period.

Methods of the invention can be used to obtain profiles ranging from high-resolution profiles (e.g. continuous, second-by-second, minute-by-minute, or hour-by-hour resolution) to low-resolution profiles (e.g. daily, weekly, or monthly resolution). Different levels of resolution can be selected for different portions of the available information. For example, in one embodiment a low resolution blood-glucose level profile is obtained for the period from 120 days to 60 days prior to the blood sample analysis, and a high resolution blood-glucose level profile is obtained for the period from 60 days to 0 days prior to the blood sample analysis. Different levels of resolution can be obtained for any portion of the available data. In addition, methods of the invention do not require that all the available data be analyzed. Indeed, a portion of the data can be analyzed in order to focus on a specific time period within a longer time period for which data is available.

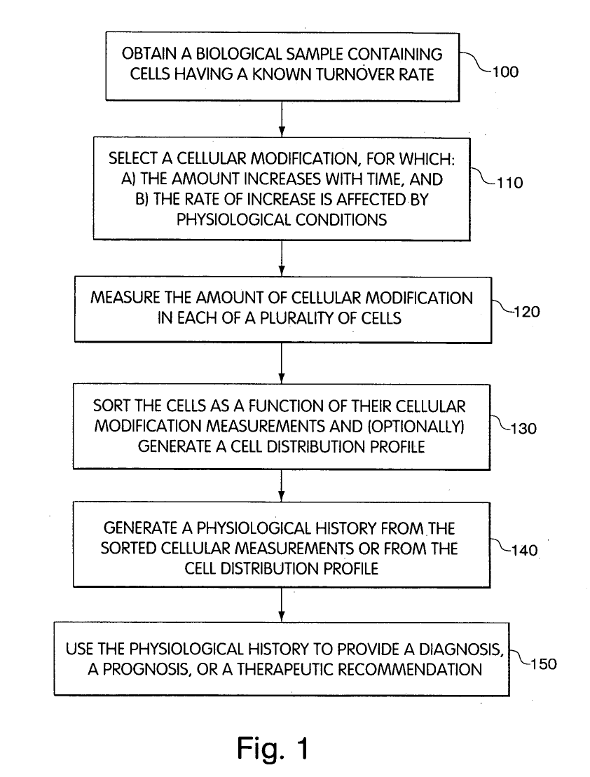

The block diagram of FIG. 1 shows an embodiment of the invention where a biological sample is obtained in step 100; a specific cellular modification to be measured is selected in step 110; the amount of the cellular modification is measured for each of a plurality of the cells in step 120; cells are sorted as a function of their cellular modification measurements in step 130, and, optionally, a cell distribution profile (CDP) is generated to represent the amount of cells as a function of the amount of cellular modification; a physiological profile is obtained in step 140 by analyzing the sorted cellular measurements or CDP of step 130; and a diagnostic, prognostic, or therapeutic determination is made in step 150 based on the physiological profile from step 140. Step 130 preferably involves ordering the raw cellular modification data as a function of either increasing cellular measurements or decreasing cellular measurements. In some embodiments, the raw data will provide a different measurement for each cell. Accordingly, the individual cells can be ordered as a function of increasing or decreasing cellular modification amounts to provide sorted cellular data. In other embodiments, the raw data will provide ranges or values of modification measurements and indicate a number of cells associated with each range or value. These groups of cells can also be ordered as a function of increasing or decreasing cellular modification amounts to provide sorted cellular data. The sorted cellular data can be analyzed directly as described herein. Alternatively, the sorted cellular data can be grouped to generate a specific cell distribution profile as described herein. The analysis of step 140 can be based on either the sorted cellular data or the CDP. In alternative embodiments, methods of the invention can be used to screen candidate drugs for desirable physiological properties or to evaluate drugs or candidate drugs for side effects including toxic side effects.

These and other aspects of the invention are described in more detail herein.

Biological Samples.

In some preferred embodiments, a biological sample includes cells that have a known lifespan. In other embodiments, a biological sample includes an antibody, a peptide, a protein, a nucleic acid, or a lipid. Examples of biological samples include blood, and particularly red blood cells from blood. Examples of cells with a known lifespan include red blood cells (an average lifespan of 120 days in male humans and 110 days in female humans). In general, a useful cellular lifespan can be of any length, ranging from days to years, but preferably on the order of several weeks or several months. It is important that the turnover rate of a cell type be sufficient to allow an amount of a cellular modification to be correlated with the age and physiological history of an individual cell. Preferred cells have a constant turnover rate and a lifespan that is similar to the length of time over which a physiological profile is to be analyzed. However, methods of the invention can be used even if the cell type being analyzed does not have a constant rate of cell generation or death. For example, the rate of cell generation or cell death may be affected by physiological changes associated with external or internal factors. Alternatively, the rate of cell generation and/or cell death may be regular, but not constant. For example, the rate of cell generation and/or cell death may undergo a cyclical change. Depending on the nature and the significance of any changes in the rate of cell generation and/or death, methods of the invention can either ignore these changes, or account for these changes by including a correction factor in one or more analytical steps. A varying cell generation rate can in some cases be detected in the analytical steps. For example, if multiple daily changes in blood glucose levels are being retrieved, the delineation of a particular day may be inferred by assuming that the subject does not eat for long periods corresponding to the sleep intervals where there are no sudden rises in glucose level like those expected soon after ingesting a meal. The cell generation can be assumed to be constant within each day. This analysis can correct for a slowly varying generation rate that is sufficiently constant over the period of a single day.

A biological sample may be processed to isolate cells of a specific cell type of interest for subsequent analysis. In some embodiments, different cell types can be analyzed together. Preferably, if multiple cell types are being analyzed together, the different cell types have similar properties with respect to lifespan and cellular modification. Alternatively, the detection method is selected to detect only cellular modifications of one cell type. In some embodiments, only one of the cell types present in the biological sample may undergo the cellular modification being measured. Analytical methods of the invention are useful even if cellular modifications are being measured on individual cells from different cell types with different turnover rates and/or modification kinetics. However, additional correction steps may be required to account for the presence of different cells types in the assay.

In general, the biological sample is obtained in such a way that a representative number of intact cells is preserved for measurement step 120. According to the invention, intact cells are cells that preserve a sufficient amount of their structural and/or functional features to provide useful information about their age and physiological history. Accordingly, a cell is considered intact if a meaningful amount of a selected cellular modification can be measured from that cell. In preferred embodiments, whole cells are analyzed. The condition of a cell required for an analysis of the invention will depend on the nature of the modification being measured. For example, a cell only need retain its membrane if a membrane-associated modification is being measured. In some embodiments, measurements of certain molecular modifications do not require intact cells.

General methods for preserving intact cells are useful for the invention, and include using isotonic solutions to preserve overall cell structures.

Preferred sizes of sample are 0.1 ml, 0.2 ml, 0.3 ml, 0.4 ml, 0.5 ml, 0.6 ml, 0.7 ml, 0.8 ml, 0.9 ml, 1 ml, 2 ml, 3 ml, 4 ml, 5 ml, 6 ml, 7 ml, 8 ml, 9 ml, 10 ml, 15 ml, 20 ml, 25 ml, 30 ml, 35 ml, 40 ml, 45 ml, 50 ml, 55 ml, 60 ml, 65 ml, 70 ml, 75 ml, 80 ml, 85 ml, 90 ml, 100 ml, 150 ml, 200 ml, 300 ml, 400 ml, 500 ml, 600 ml, 700 ml, 800 ml, 900 ml, 1000 ml, or more.

Preferred numbers of cells analyzed include: 10-10,000,000 cells, preferably 100-1,000,000 cells, and more preferably 200 to 100,000 for each time interval that is retrieved in the analysis. Accordingly, depending on the number of time intervals being analyzed and the desired resolution of the retrieved physiological history, the total number of cells being analyzed can vary from less than 1,000,000 to more than 100,000,000,000. In many embodiments, about 10,000,000,000 cells are analyzed to obtain an approximately 110 or 120 day blood glucose history for a subject. In one embodiment, between about 20,000 and about 1,000,000 cells, and preferably about 1,000,000 cells are analyzed to obtain weekly measurements (about 17 bins corresponding to about 17 weeks) for a subject. In another embodiment, between about 180,000 and about 8,000,000 cells, and preferably about 8,000,000 cells are analyzed to obtain daily measurements (about 120 bins corresponding to about 120 days) for a subject. In another embodiment, between about 4,500,000 and about 200,000,000 cells, and preferably about 200,000,000 cells are analyzed to obtain hourly measurements (about 2,880 bins corresponding to about 2,880 hours) for a subject. In another embodiment, between about 250,000,000 and about 10,000,000,000 to 15,000,000,000 cells, and preferably about 10,000,000,000 to 15,000,000,000 cells are analyzed to obtain minutely measurements (about 172,800 bins corresponding to about 172,800 minutes) for a subject.

Preferred samples include biological fluids such as blood, serum, lymph, sperm, sputum, intraperitoneal fluid, cerebrospinal fluid, lacrimal fluid, saliva, urine, vaginal fluid. Other samples include tissues and biopsies such as, for example, cancer tissue.

Cellular Modifications.

In general, useful cellular modifications include modifications for which a) the total amount of modification of an individual cell increases with the age of the cell type being analyzed; and b) the rate of modification is affected (either increased or decreased) by changes in a physiological variable of interest. Generally useful cellular modifications include modifications that can be measured for individual cells or small groups of cells. The measurements can be used as a basis for sorting the cells as described herein.

According to the invention, a cellular modification can be on a cell surface or inside the cell, provided that the cellular modification confers a measurable property on the cell. Preferably, the amount of the measurable property is directly correlated with the actual amount of the cellular modification.

Cellular modifications include modifications of any one or more cell constituents, including proteins, lipids, nucleic acids (RNA and DNA), and metabolites. Any one or more of these constituents can be on the cell surface, cell-membrane bound, organelle-membrane bound, nuclear-membrane bound, endoplasmic-reticulum membrane bound, soluble, cytosolic, nuclear, organelle, monomeric, multimeric, or part of a large complex. Preferred modifications are irreversible. Preferred modifications include alkylation (e.g., methylation), sulfonation, non-enzymatic modifications including non-enzymatic glycation, enzymatic modifications including enzymatic glycosylation, other modifications, and combinations thereof.

Hemoglobin glycation is an informative cellular modification for analyzing a subject's red blood cells, because the total amount of hemoglobin glycation on a red blood cell increases with the age of the red blood cell, and the rate of hemoglobin glycation is affected by the blood glucose level in a subject. The rate of glycation increases with increased hyperglycemia (provided that the glucose level does not rise above an upper threshold beyond which no appreciable increase in the glycation rate is observed) and decreases with increased hypoglycemia (provided that the glucose level does not drop below a lower threshold beyond which no glycation is observed). As used herein, hemoglobin glycation refers to glycation of any one or more of hemoglobin A, hemoglobin A2, hemoglobin F, hemoglobin C, or hemoglobin S. In preferred embodiments, glycation of hemoglobin A is measured. However, glycation of any hemoglobin type or combination thereof can be measured. In some embodiments, glycation of one or more other molecules can be measured.

Methods for Measuring the Amount of Cellular Modification of a Plurality of Individual Cells in a Sample.

In general, useful methods include methods that have sufficient resolution and sensitivity to measure individual cellular modification amounts. Preferred methods have a sufficiently high throughput rate to measure individual cellular modification amounts for large numbers of cells. Useful throughput rates are greater than 100 cells per minute. However, throughput rates can be greater than 1000; 10,000; 100,000; 1,000,000; 10,000,000; 100,000,000; or 1,000,000,000 cells per minute.

According to the invention, a measurement can be an absolute measurement or a relative measurement. An absolute measurement provides a value for the total amount of cellular modification of a given cell. A relative measurement provides a measure of the amount of cellular modification of one cell relative to the amount of cellular modification of one or more other cells.

There are two general approaches for measuring an amount of cellular modification of individual cells in a cell sample. In a first approach, cells are first separated and then measured. In one embodiment, cell solutions can be diluted, e.g. serially diluted, to obtain fractions containing one cell on average. General methods and devices for automated serial dilution can be used, for example chambers or multi-well plates can be used. In another embodiment, a single cell can be obtained in a capillary pipette by microsuction using a microinjector, for example. This cell can then be assayed in the pipette. The cell can also be deposited in a reaction volume (e.g. in a reaction tube or in a well on a multi-well plate) for further analysis. The cell can also be analyzed by mass-spectrometry, using a method such as single-cell matrix-assisted laser desorption/ionization time-of-flight (MALDI-TOF) described in Shimizu et al. (February, 2002), Analytical Sciences, vol. 18, pp. 107-108; and Shimizu et al. (January, 2003), Analytical Sciences, vol. 19, pp. 49-53. Each isolated cell can also be analyzed using any appropriate method. For example, a single RBC can be analyzed using an HbA1c type measurement to determine its amount of glycated hemoglobin. Other assays, including immunological assays (e.g. an ELISA or using detectably labeled antibodies, other binding agents or ligands such as fluorescently-labeled or radio-labeled reagents). The analysis can be repeated for as many cells as are required for the information that is sought to be retrieved. Preferably, between 100 and 100,000 to 1,000,000 cells are analyzed this way. However, in some embodiment more cells can be analyzed.

In a second approach, cells are separated or sorted as they are measured. For example, in a cell sorter the cells are sorted as a function of the amount of cellular modification of each cell.

In general, for either approach, the amount of modification can be detected directly (e.g. the cellular modification itself changes a detectable property of the cell, for example fluorescence, charge, mass, size, or other property) or indirectly (e.g. the cellular modification is tagged or labeled using a reagent such as a reagent conjugated to a fluorescent dye, a radio-label, a molecular-weight-tag, a size-tag, an enzyme, an electrostatic-moiety, and magnetic-moiety, an electromagnetic moiety, or other detectable moiety).

Some generally useful methods include cell sorting, mass spectrometry, chromatography, and electrophoresis.

In some embodiments, the cellular modification results in a property that can be directly measured. In other embodiments, the cellular modification can be exposed to a reagent that produces a property that is measured. The nature of the property being measured depends on the measurement method. The property can be one of the following properties: physical size, mass, electrostatic, electromagnetic, fluorescent, enzymatic, hydrophobic, reactivity, or other property that is measurable (either directly or indirectly).

Cell sorters (such as those described in U.S. Pat. Nos. 5,158,889; 6,120,735; and 6,540,895, the disclosures of which are incorporated herein by reference) can separate cells as a function of a number of different properties. However, a common basis for cell sorting is fluorescence intensity using a fluorescence-activated cell sorter (FACS). If the cellular modification amounts do not result in measurable differences in fluorescence, the cellular modification can be labeled using a fluorescent marker. Useful markers include one or more fluorescently labeled antibodies, where the antibody is specific for the cellular modification. For example, glycated hemoglobin can be detected using a specific monoclonal antibody for glycated hemoglobin (e.g. an IgG specific for human glycated hemoglobin such as clone number M511819 available from Fitzgerald Industries International, Inc., Concord, Mass.). Glycated hemoglobin can also be labeled using a polyclonal antibody, or other binding agent or ligand that specifically binds to glycated hemoglobin. In some embodiments, the amount of non-glycated hemoglobin can be measured in a cell, and the amount of glycated hemoglobin can be calculated from the total amount of hemoglobin in the cell. For example, non-glycated hemoglobin can be labeled using a monoclonal, polyclonal, or other binding agent or ligand that specifically binds to hemoglobin. Antibodies and other binding agents or ligands can be conjugated to a labeled moiety using known methods and reagents. For example, methods for conjugating antibodies to fluorescent dyes include chemical methods known in the art. Useful fluorescent dyes include Fluorescein (FITC), Phycoerythrin (PE), Cy5PE, Cy7PE, Texas Red (TR), Allophycocyanin (APC), Cy5, Cy7APC, Cascade Blue, Biotin.

Chromatography can separate cells on the basis of any one or more of a number of different properties including size, charge, and hydrophobicity. Different types of chromatography resin or gel are used for different types of separation. If the amounts of cellular modification cannot be directly distinguished using chromatography, a property useful for chromatography can be conferred on a modified cell using a specific reacting or binding agent such as an antibody, preferably conjugated to an additional moiety.

Mass spectrometry can separate cells on the basis of mass. If the amounts of cellular modification alone do not result in significant differences in cellular mass, the cellular modification can be tagged with a molecular weight marker. For example, a specific antibody attached to a molecular weight marker can be used.

Electrophoresis can separate cells on the basis of charge and or size. Again, if the cellular modifications alone are not sufficient to confer detectable differences on the cells, specific binding reagents can be used to tag the modified cells. In some embodiments, the cells can be treated with a reagent that specifically reacts with the cellular modification to produce a measurable property such as change in charge on the cells.

Whatever method is used, the data points can be converted into amounts of cellular modification. Typically, cells with known cellular modification at one or more data points corresponding to one or more known amounts of cellular modification (e.g. 0%, 50%, or 100%) can be used to calibrate the results. A 5% reference data point can be produced by exposing part of the sample to glucose for several days (e.g. 925 mg/dL for seven days) and then measuring the glycation of the least glycated cells. These are cells that were 0% glycated before the in-vitro exposure to glucose. In many embodiments, the youngest cells in a sample are the least modified. In a blood sample, for example, the least glycated cells are typically the youngest cells. Accordingly, a blood sample may contain very young cells that do not yet contain glycated hemoglobin. However, depending on the sample and the patient, the least glycated cells may contain a small amount of glycated hemoglobin. In addition, depending on the device used to analyze the cells, it may be difficult to accurately quantify the cells with the lowest amount of glycated hemoglobin. Nonetheless, the lowest detectable level of glycation can be used as a reference point representing the youngest cells in the sample. In some embodiments, the least glycated cells in the sample can be used as a 0% reference point. However, in other embodiments, the lowest level of detectable glycation can be between 0%-10%, 0.1%-5%, or 5%-10%.

Alternatively, the performance characteristics of an apparatus used to analyze cells is known. In some embodiments, the data points obtained from the cellular analysis can be directly converted into cellular modification amounts using a known (or calculated) conversion factor. However, some information may be required from each subject (e.g. average cell size average amount of hemoglobin in a subject's red blood cell, number of red blood cells per volume of blood) in order to account for subject to subject variations.

In some embodiments, an HbA1c measurement can be performed to calibrate the conversion from the data points to amounts of cellular modification. The amount of cellular modification measured from an HbA1c measurement should be the same as the total amount of cellular modification obtained by summing all the cellular modification values calculated from the raw data points (correcting for any difference in the number of cells used for the different assays if necessary).

The raw data points or the resulting cellular modification values can be stored or archived in a retrievable form in a computer memory or on a computer-readable medium, tabulated, and/or plotted graphically to show the absolute or relative measurement for each cell.

The data includes the youngest cells at the low end (least modified) and the oldest cells at the high end (most modified). The width of the total modification range (of either the raw data points or the cellular modification values) therefore represents the time period that can be analyzed. For example, for blood glucose analysis, the width of the hemoglobin glycation data can represent 120 days (the life span of a red blood cell in a human male) or 110 days (the life span of a red blood cell in a human female). Accordingly, analytical methods of the invention can be corrected for a subject's gender, age, or disease status, by using an appropriate value for the total time period that is represented by the total width of the cellular modification data.

According to the invention, an amount of cellular modification can be an absolute amount or a relative amount (e.g. a % or ratio) of cellular modification.

In some embodiments, a fraction of the red blood cells is isolated prior to analysis by FACS. For example, a fraction of the most recent blood cells can be isolated as those cells with the least amount of glycated hemoglobin (e.g. between 1 and 20% of the most recent cells can be isolated). Alternatively, a fraction of older cells can be isolated with the most amount of glycated hemoglobin (e.g. between 1 and 20% of the oldest cells can be isolated). These cells can be isolated for example by chromatography or electrophoresis as described herein.

Methods for Obtaining a Cell Distribution Profile from a Measurement of the Amount of Cellular Modification of a Plurality of Individual Cells in a Sample.

In general, the process of obtaining a cell distribution profile involves sorting the cells as a function of the measured amount of cellular modification for each cell. In some embodiments, the sorted cells are further divided into ranges to create a curve that represents the number of cells as a function of cellular modification.

In theory, nearly every cell may have a unique amount of cellular modification. Therefore, a plot of the sorted cells could be an essentially binary or digital plot with either one cell or zero cells for any given amount of cellular modification. However, in practice, depending on the resolution of the measurement step, the sorted cells may already fall into ranges of cellular modification values, as opposed to having a unique measurement for each cell.

In either case, a cell distribution profile can be created from the sorted cell data. This step generally involves dividing the total width of the raw data points or the cellular modification values (absolute or relative) into a series of smaller ranges of cellular modification. These ranges preferably form a subset of consecutive ranges within the width of cellular modification values. The ranges do not have to represent equal relative changes in the amount of cellular modification. However, in preferred embodiments, each smaller range represents an equal relative change in cellular modification. The size of the ranges is preferably chosen so that a) the range size is large enough for each range to include at least one cell and preferably about 2-5; 10; 15; 20; 25; 30; 50; 100; 500; 1,000; 5,000; 10,000; 50,000; 100,000; 500,000; 1,000,000; 5,000,000; 10,000,000; 50,000,000; 100,000,000; 500,000,000; or 1,000,000,000 or more cells, and b) the range size is small enough so that it is smaller than a range corresponding to a unit of time being represented with a measurement in the retrieved history. Preferably, in order to preserve the historical information that is present in the data, the ranges are small enough so that several ranges (2-10; 10-20; 20-50; 50-100; 100-500; 500-1,000 or more) are required to obtain a single historical event of interest in the analytical steps. Therefore, the width of the initial data is preferably divided into about 10-100; 100-1,000; 1,000-10,000; 10,000-100,000; 100,000-1,000,000; 1,000,000-10,000,000; 10,000,000-100,000,000; 100,000,000-1,000,000,000 or more equal ranges.

The size of each of the small ranges affects the resolution of the subsequent analysis. Preferred ranges for glycated hemoglobin are between 0% and 50% and more preferably between 0% and 30% hemoglobin glycated. The size of each small range preferably corresponds to a glycation difference of between about 0.001% and 0.1%. However, the size of each small range can be larger or smaller. In some embodiments, the size of the small range represents a glycation difference of about 0.002%, 0.003%, 0.004%, 0.005%, 0.006%, 0.007%, 0.008%. 0.009%, 0.01%, 0.2%, 0.03%, 0.04%, 0.05%, 0.06%, 0.07%, 0.08%, 0.09%, or 0.01%. Preferred total width of glycated hemoglobin is between the following values 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0% hemoglobin glycated.

The resulting cellular distribution profile can be stored or archived in a retrievable form in a computer memory or on a computer-readable medium, tabulated, and/or plotted graphically to show a number of cells as a function of cellular modification amount. The number of cells can be provided as an absolute number, a percentage of the total number of cells, or a measurement relative to a reference amount of cells. The invention is not limited in this respect.

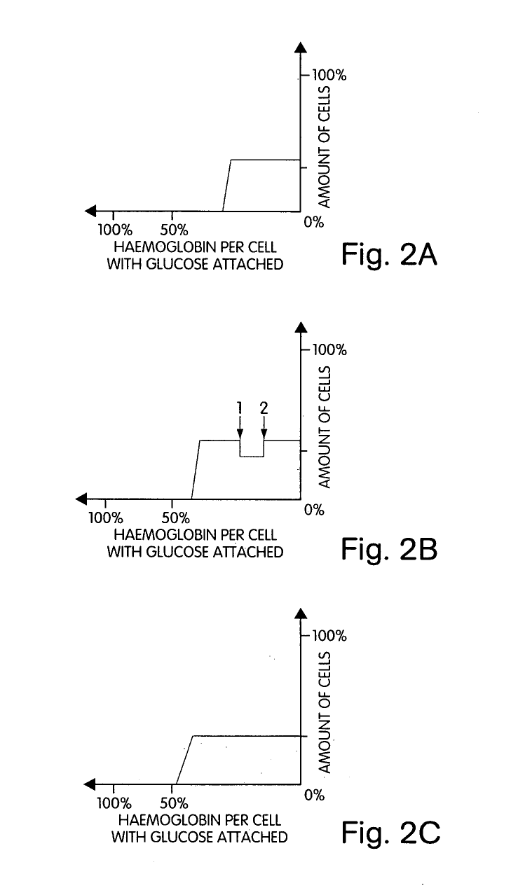

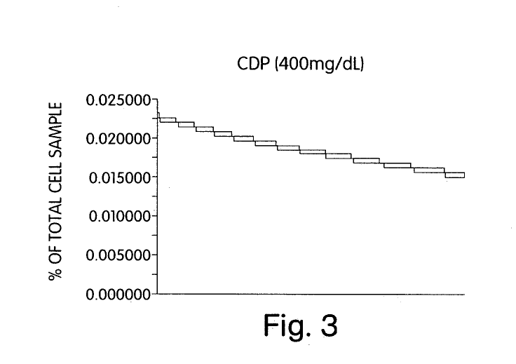

The sorted cellular measurements and the CDP can be represented graphically in the form of a Figure such as FIG. 2 or FIG. 3. FIGS. 2 and 3 show relative amounts of cells (% cells from the sample being analyzed) as a function of the amount of hemoglobin glycation. The sorted cellular measurements and the CDP can be based on the observed number of cells in the range(s) of interest, or on the relative number of cells (e.g. percentage or fraction of observed cells) in the range(s) of interest.

In FIGS. 2 and 3 the data is shown with the amount of hemoglobin modification increasing from right to left on the x axis. This representation is useful, because the amount of modified hemoglobin on a cell is a reflection of both the age of the cell and the physiological history of the cell. Therefore, older cells are towards the left of the figure and younger cells are towards the right. Therefore, the x axis includes a time component in addition to a physiological component.

FIG. 2A) shows a normal subject. FIG. 2B) shows a subject that has suffered an event leading to increased glucose levels at about position 1 followed by a treatment that returns the glucose to approximately normal levels at about position 2; FIG. 2C) shows a diabetic patient that has not been treated over the time represented by the analysis.

FIG. 3 shows a more detailed profile of FIG. 2C). In FIG. 3 the amount of cells as a function of hemoglobin modification is not linear. The relative amount of cells increases with the amount of glycation. This represents the anticipated effect of a decreased glycation rate as the percentage of glycated hemoglobin molecules in a cell increases.

The orientation of the axes in these figures can be changed as the invention is not limited in this respect.

In some embodiments, the sorted cellular measurements and/or CDP can be represented graphically in any orientation, as a bar graph, a column graph, or any other form of graphic representation as the invention is not limited in this respect.

The cell distribution profile provides an intuitive or approximate general overview of the physiological history of a subject. However, this data can be analyzed further to obtain a precise profile of the subject's physiological history as a function of time.

Analytical Methods for Obtaining a Physiological History for a Subject.

In general, a physiological history can be obtained by dividing the sorted raw data points representing the measured amounts of cellular modification (values or ranges) and/or a CDP into “bins,” each bin including a plurality of cells corresponding to a time period. A bin with more cells represents a greater period of time than a bin with fewer cells. Preferably, the bins are chosen to represent about 1 minute, 1 hour, 6 hours, 12 hours, 1 day, one week, or one month. Preferably, each bin contains the same number of cells. If the cell production rate is constant, equal numbers of cells will represent equal time periods. Therefore, if the original width corresponds to 120 days, and the data is divided into 120 bins containing equal numbers of cells, then each bin corresponds to one day.

In general, a CDP (or other sorted data) can be analyzed to obtain a physiological profile that corresponds to the entire time range or a fraction of the time range for which information is available. The length of time for which information is available is a function of the lifespan of the cell being studied, as discussed above.

In addition, the CDP (or other sorted data) can be analyzed to obtain profiles with different levels of resolution.

Different analytical approaches can be used to obtain a physiological profile as a function of time. Although the systems and methods described below are described primarily in relation to blood glucose level analysis, the systems and methods described herein are not limited thereto, but may be applied to other situations, and other analytical approaches can also be used as the invention is not limited in this respect.

a) Subdivide the CDP (or Other Sorted Data) into a Series of Bins

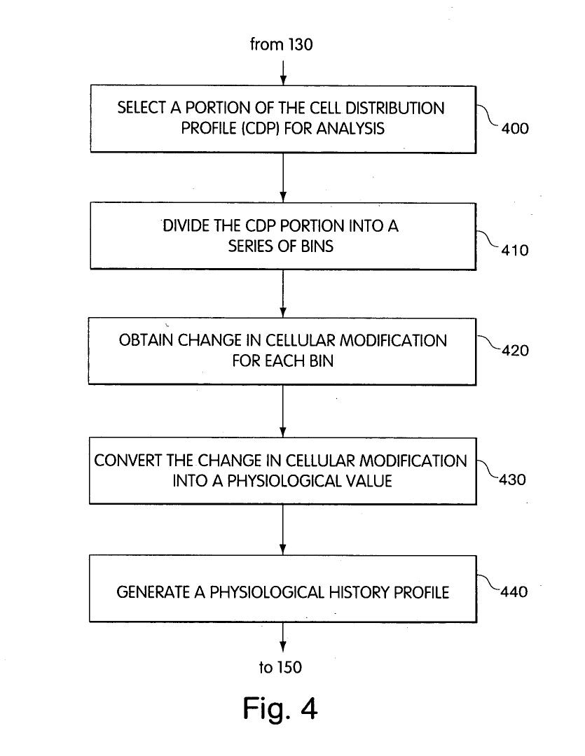

The block diagram of FIG. 4 shows an embodiment of this method. This represents an embodiment of step 140 in FIG. 1. In step 400, a portion of the CDP (or other sorted data) is selected for analysis. The size of the portion selected will depend on the target time of interest. In step 410, the CDP (or other sorted data) is divided into a series of bins. The size of the bins are chosen to correspond to the time unit of interest. In step 420, the change in the amount of cellular modification across each bin is calculated. In step 430, the calculated amount of change for each bin is converted into a physiological value that would account for the calculated change.

In alternative embodiments, a value associated with a first bin can be compared with a value associated with a second bin to obtain a measure of the change in cellular modification between two bins. This measure can then be converted into a physiological value. Other approaches for calculating a change in cellular modification between time points of interest can also be used as the invention is not limited in this respect. In general, for all of these analyses, an amount of change within bins or between bins is calculated. The calculated change is then converted into a physiological parameter based on a conversion factor that is a function of the size and nature of the bins. Additional correction factors may also be required.

An embodiment of this approach is discussed in more detail in the following paragraphs. The following discussion relates to an example using a CDP representing the number of cells as a function of glycated hemoglobin. However, this method is generally applicable to other cellular modifications and other forms of sorted data.

Preferably, the CDP is subdivided into a series of bins such that each bin contains an equal number of cells and therefore represents a discrete amount of tine (e.g. a day), assuming that the cell production rates are constant over the time period being analyzed.

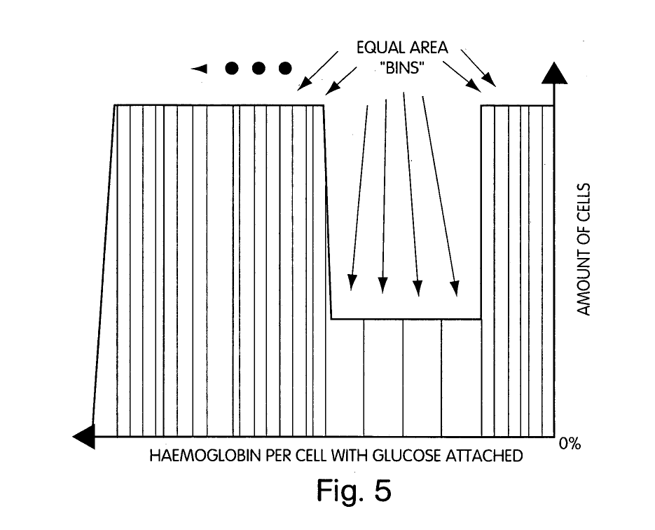

FIG. 5 shows an embodiment where the CDP is divided into days. Taking one reading per day, there are 120 “bins”. If the patient's average RBC lifespan is more or less than 120 days, the results can be corrected in a subsequent step as described herein. The difference in the percentage of glucose from the left edge to the right edge of each bin is assumed to be linearly proportional to the glucose level on that day. Corrections can be made for non-linearity due to slowing of glycation rate in the leftmost bins (the bins with the most amount of glycation in this example). In general, the non-linearity of the glycation rate is present at all time points. Preferably, analytical methods of the invention correct for this non-linearity at all time-points. However, the effect of non-linearity is more pronounced in cells with more glycation (in the left-most bins in FIG. 5). All of the numbers with regard to cell counts are bins of cells with close to the same amount of glucose bound as discussed above. It will be rare (if ever) for two cells to have exactly the same percentage of glycated hemoglobin. FIG. 5 indicates the occurrence of a pronounced glucose level change, as well as the time period after a treatment was initiated.

Such an analysis can be performed using a computer algorithm. An embodiment of a computer algorithm is provided in Example 2. However, other algorithms can also be used. In general, once the target bin size is determined, the number of CDP ranges required to make each bin is obtained. In preferred, embodiments, each range represents an equal change in modification. However, each range typically contains a different number of cells. Therefore, different numbers of ranges will be required to complete different bins (even though each bin is preferably chosen to represent a same amount of tine and therefore contain the same total number of cells). Therefore, different bins will be associated with different changes in modification. The change in modification represented by a bin can be calculated in any way as the invention is not limited in this respect. In preferred embodiments, the change in modification for a given bin is calculated as the difference in glycation between the leftmost CDP range in the bin and the rightmost CDP range in the bin.

In alternative embodiments, the CDP can be divided into a series of bins with equal ranges of glycosylation per bin. The analysis is similar to the analysis described above. However, each bin represents an expected day, and would contain approximately the same number of cells if the blood glucose levels had been constant over the time period being analyzed. Differences in numbers of cells per bin represent changes in glycation rate and can be used to calculate the blood glucose levels in the subject over the time period being studied.

Such an analysis can also be performed using a computer algorithm.

In yet further embodiments, the CDP can be divided into a series of bins based on other parameters. The analysis is based on similar principles as discussed above, and may require additional steps to account for the parameters used to subdivide the CDP.

In all of the analyses described above, the CDP could have been divided into different time periods. Alternatively, portions of the CDP could have been used in order to focus on specific time periods.

In some embodiments, the amount of non-glycated hemoglobin in each cell can be measured in addition to the amount of glycated hemoglobin. Accordingly, the ratio of glycated to non-glycated hemoglobin can be directly calculated. In other embodiments, the amount of non-glycated hemoglobin is not measured for each cell. A ratio of glycated to non-glycated hemoglobin can be determined from a measurement of the amount of glycated hemoglobin by assuming a constant amount of total hemoglobin in each cell.

b) Obtain a Numerical or Graphical Representation of the CDP and Compare it to a Reference CDP

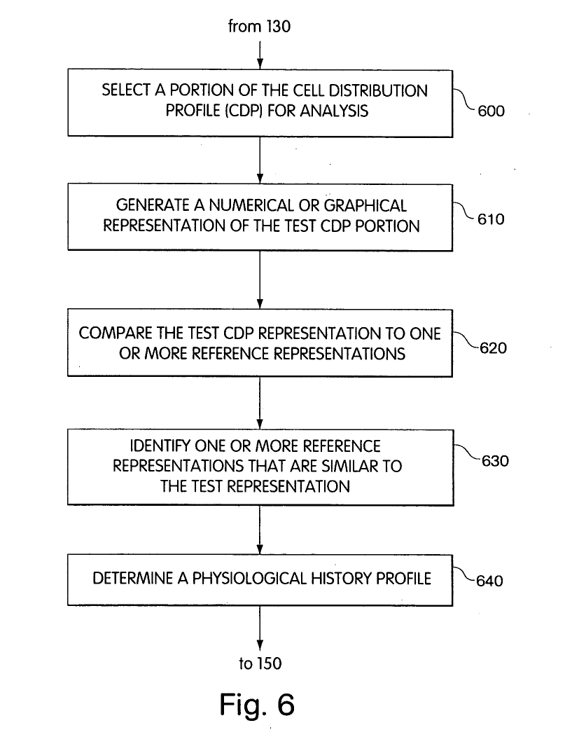

The block diagram of FIG. 6 shows an embodiment of this method. This represents a further embodiment of step 140 in FIG. 1. In step 600, a portion of the CDP (or other sorted data) is selected for analysis. The size of the portion selected will depend on the target time of interest. In step 610, a numerical or graphical representation of the CDP (or other sorted data) is generated. In step 620, the representation from step 610 is compared to a reference representation. In step 630, one or more reference representations is identified as similar to the test representation. In step 640, a physiological history is determined from the known physiological history associated with the similar reference representation(s) identified in step 630.

c) Mathematical Analysis

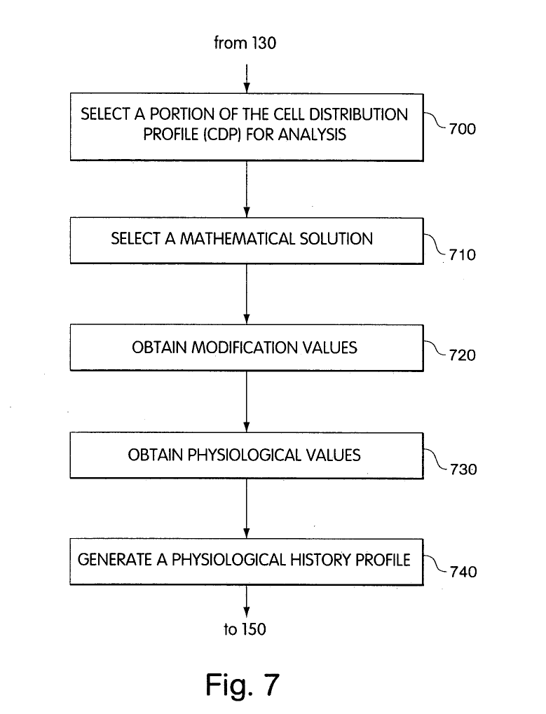

The block diagram of FIG. 7 shows an embodiment of this method. This represents a further embodiment of step 140 in FIG. 1. In step 700, a portion of the CDP (or other sorted data) is selected for analysis. The size of the portion selected will depend on the target time of interest. In some embodiments, the entire width of the CDP (or other sorted data) is analyzed. In step 710, a mathematical solution is selected. In step 720, values for the change in cellular modification are obtained (either as a function of time or as a function of the amount of cellular modification of each cell). In step 730, the values from step 720 are converted to physiological values corresponding to the calculated values for the change in cellular modification. These steps can be performed using a computer and an appropriate algorithm.

In one embodiment, the following equations can be solved to obtain blood glucose values.

In general, the glucose (g) level as a function of “sample point” time (tₛₚ) can be written as follows when tₛₚ<0. In preferred embodiments, the time is set at 0 when a sample is removed from a subject, and the past history of the subject is analyzed and presented as negative times prior to time 0. However, similar mathematical analyses can be performed using different time reference points (e.g. using the oldest available time as time 0 and using positive times after time 0):
g(tₛₚ)=K_{b}·Δ Equation (2)
Where
   Δ is a measure of the difference in hemoglobin glycation over a unit of time centered on tₛₚ; and,
   Kb is a constant that relates the value of Δ to a glucose level known to be associated with the observed change in glycation over time.

Values for Kb can be obtained experimentally. Values for Kb can also be calculated as discussed below.

The value for Δ as a function of Xₛₚ (the amount of glycation at time tₛₚ) can be obtained by solving the following equation for Δ:

$${\int }_{Xsp - \frac{Δ}{2}}^{Xsp + \frac{Δ}{2}} f ( x ) ⅆ x = {K}_{a} \qquad Equation ( 3 )$$

Where
   Ka represents the cells in a window of time that is selected for the analysis (e.g. one minute, one hour, or one day); and,
   f(x) is the CDP function.

Ka therefore represents a number of cells corresponding to the selected window of time for which g(tₛₚ) is being calculated.

The value for x at time tₛₚ can be obtained by solving the following equation for Xsp:

$${t}_{sp} = - L \cdot \frac{{\int }_{Xsp}^{0} f ( x ) ⅆ x}{{\int }_{100 \% ( glycated )}^{0} f ( x ) ⅆ x} \qquad Equation ( 4 )$$

Where L is the life span of a red blood cell (L corresponds to the width of the data, and represents other widths in other embodiments).

In preferred embodiments, the rate of glycation can be corrected to account for changes in the rate of glycation as a function of the number of glycated hemoglobin molecules in a cell. The corrected rate of glycation r(tsp) can be obtained from the following equation:

$$r ( {t}_{sp} ) = \frac{100 \%}{100 \% - {x}_{sp}} \cdot Δ \qquad Equation ( 5 )$$

To obtain the glucose level g at time tsp, the corrected rate of glycation r(tₛₚ) can be substituted for Δ in Equation (2) above.

In alternative embodiments, g(tsp) can be obtained by solving either one of equations (6) or (7) for g(tₛₚ):
r(tₛₚ)=Ke·(1−K_{c}^{(Kd·g(tsp))}) Equation (6)
g(tₛₚ)=(1/K_{d})·log K_{c}(1−(r(tₛₚ)/Ke)) Equation (7)

Where K_{c}, K_{d}, and Kₑ are constants. K_{c} is a probability of glucose not binding to Hb. K_{d} is a scaling factor that provides the number of glucose molecules for a given glucose concentration. K_{d}.g(tₛₚ) is the number of glucose molecules. Kₑ is a scaling factor that provides the rate of glycation based on the probability that glucose binds to Hb. Kₑ is the same as r(tₛₚ) at very high (saturating) glucose levels. The constants K_{c}, K_{d}, and Kₑ are not subject specific and can be obtained from the analysis of several data points for any subject. These equations account for non-linear variations in the rate of glycation as a function of glucose levels. The relative increase in glycation rates decrease as the levels of glucose increase. However, these effects might not be significant under physiological glucose concentrations, and this correction is not required in order to obtain a useful physiological profile.

The value for Kb can be obtained from the following equation:

$$Kb = \frac{{\int }_{100 \%}^{0} f ( x ) ⅆ x}{Ka \cdot L} \cdot {K}_{rate} \qquad Equation ( 8 )$$

Where Krate is approximately

$${10}^{8} \frac{mg \cdot s}{\% \cdot dL} ,$$

and % represents % glycation, time s is in seconds. Kb units are (mg/dL)/%.

In another embodiment, these equations can be solved using discrete mathematical analysis. In this embodiment, the integrals are treated as sums. However, the mathematical analysis is based on the same model.

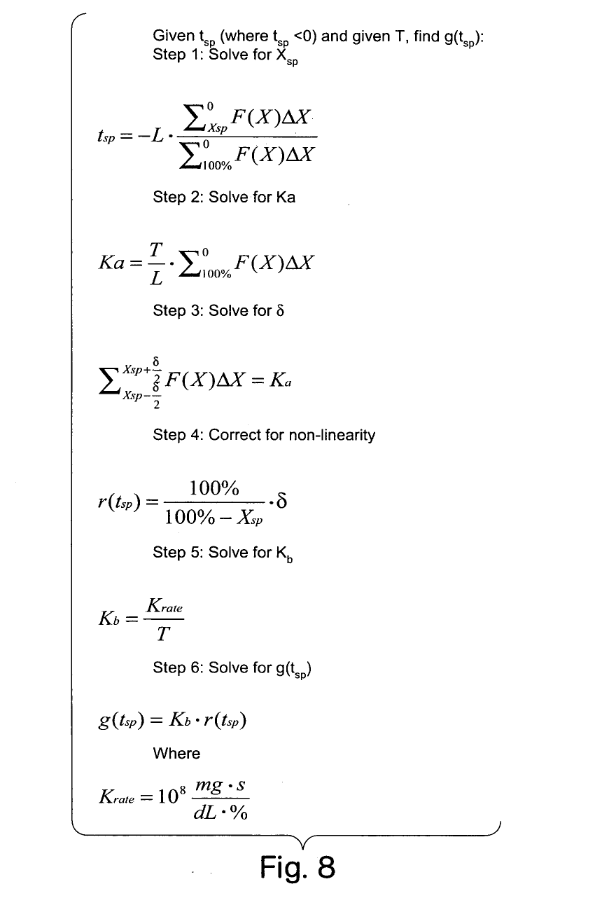

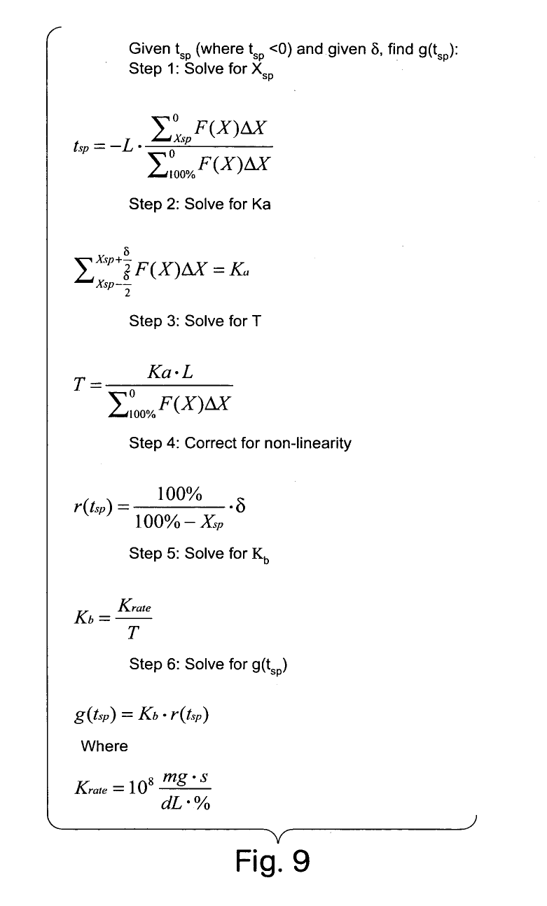

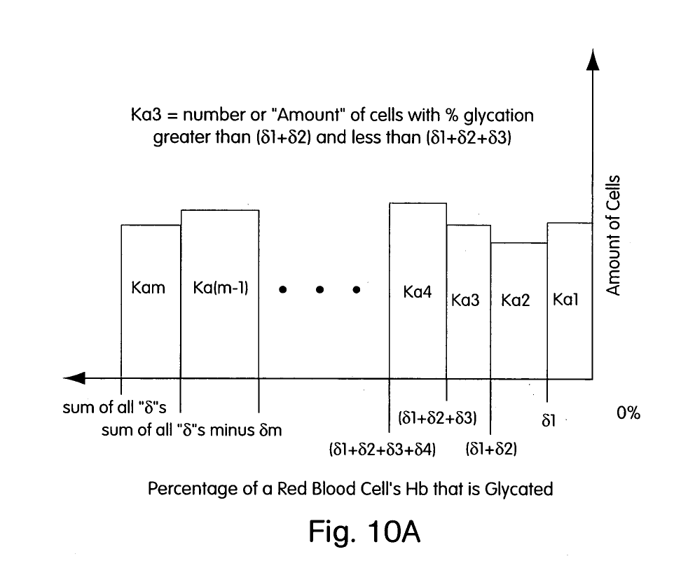

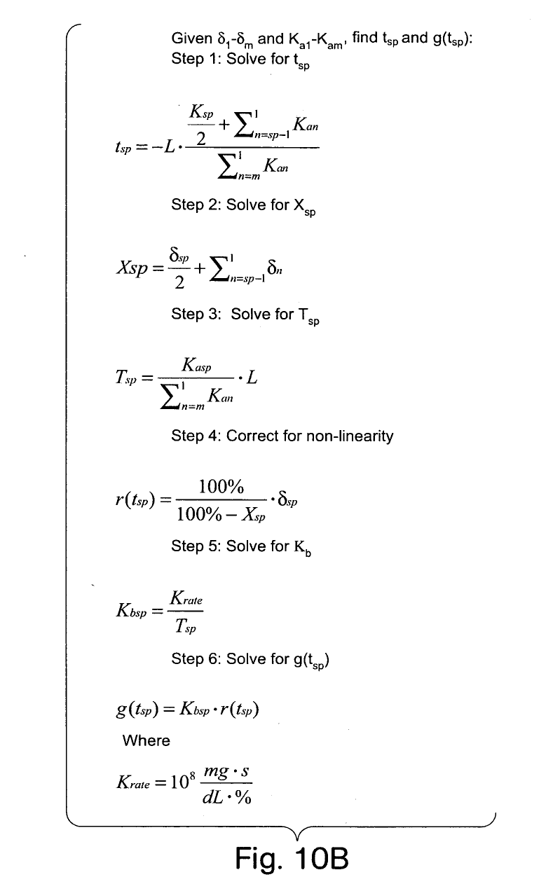

FIGS. 8-10 show examples of discrete form analysis. In FIG. 8, the time period T corresponding to a bin is chosen for the analysis. The blood glucose level g(tₛₚ) is calculated for each tₛₚ (sample point time) corresponding to a time period T as follows. In step 1, Xₛₚ (the amount of glycation corresponding to time tₛₚ) is obtained. In step 2, Kₐ (the amount of cells per time period) is obtained. In step 3, δ (the change in glycosylation corresponding to a time period T) is obtained. In step 4, the glycation change is corrected for non-linearity. In step 5, K_{b} is obtained. In step 6, g(tₛₚ) is obtained. In FIG. 9, the range of glycation 6 is provided for each bin (this range is either chosen by the operator or determined by the measurement data). The blood glucose level g(tₛₚ) is calculated using similar steps to those described above for FIG. 8. However, the T values corresponding to each 6 may be different. Accordingly, the tₛₚ time points may be unequally spaced along the subject's profile. FIG. 10 shows this analysis in additional detail. FIG. 10 shows an embodiment of an analysis where the glycation ranges (δ₁-δₘ) are provided directly from the measurement data (they are determined by the device used to measure the cellular modifications). In this embodiment, δ₁ is the rightmost range (corresponding to the youngest cells), and δₘ is the leftmost range (corresponding to the oldest cells). The corresponding number of cells (Kₐ₁-Kₐₘ) for each glycation range are also provided. The corresponding time periods and sample time points are calculated as shown and the g(tₛₚ) for each sample time point is obtained. The equations of FIG. 10 treat an individual red blood cell as if it were instantaneously created. The equations and the analysis can be altered to reflect a different rate of glycation during the formative period. The equations can also be altered to reflect a percentage of red blood cells circulating in the blood stream which are immature, that percentage being different than the percentage which are mature cells created during a previous period of time equal in length to the period of time that it takes for a red blood cell to mature. The equations and the analysis can be altered to account for changes in the number of Hb molecules and for changes in the exposure to blood (and therefore to blood glucose) during the maturation of a red blood cell.

According to the invention, there are several sources of error that can be corrected or accounted for. Sample variance can generate error. However, the effects of statistical variances in a sample can be reduced by using a large number of cells. For example, a 3 ml blood sample contains on the order of 20 billion cells. If several billion cells are used to generate an hourly profile for a subject over a 110-120 day period, the error due to statistical variance in the cell sample should be less than 1% for nearly all returned data points. However, an analysis can be performed with fewer cells, because a recovered profile is useful even if the error is larger.

Another potential source of error is the possible statistical variation in the rate of glycation. The error from this variation can be minimized by using large numbers of cells. As discussed herein, certain non-linear components of glycation-rate variation can be corrected for in the analytical steps.

A further potential source of error is the possible statistical variation in the cell production rate. This can be corrected for as discussed herein. In addition, the error associated with variable cell production rates is likely to be low. RBC production rate is relatively constant in the absence of acute trauma such as an automobile accident.

Yet another source of error is associated with the device that is used to obtain the measurements. This error is a function of the device being used, and the size of the error will be known to the operator and can be minimized by using appropriate controls and duplicate samples if necessary.

In general, the amount of error can be reduced by using two or more samples or performing two or more assays and obtaining an averaged historical profile for a subject.

In preferred embodiments, the size of the error is minimized. However, in some embodiments, an error of 5%, 10%, 15%, 20%, or higher can be tolerated.

The methods described herein, acts thereof and various embodiments and variations of these methods and acts, individually or in combination, may be defined by computer-readable signals tangibly embodied on a computer-readable medium, for example, a non-volatile recording medium, an integrated circuit memory element, or a combination thereof. Such signals may define instructions, for example, as part of one or more programs, that, as a result of being executed by a computer, instruct the computer to perform one or more of the methods or acts described herein, and/or various embodiments, variations and combinations thereof. Such instructions may be written in any of a plurality of programming languages, for example, Java, Visual Basic, C, C#, or C++, Fortran, Pascal, Eiffel, Basic, COBOL, etc., or any of a variety of combinations thereof. The computer-readable medium on which such instructions are stored may reside on one or more of the components of a system such as the system described below, and may be distributed across one or more of such components.

The computer-readable medium may be transportable such that the instructions stored thereon can be loaded onto any computer system resource to implement the aspects of the present invention discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a processor to implement the above-discussed aspects of the present invention.

It should be appreciated that any single component or collection of multiple components of a computer system, for example, the computer system described below, that perform the functions described herein with respect to the method can be generically considered as one or more controllers that control the functions discussed herein. The one or more controllers can be implemented in numerous ways, such as with dedicated hardware, or using a processor that is programmed using microcode or software to perform the functions recited herein.

Diagnosis, Prognosis, Therapy, and Drug Discovery and Evaluation.

A profile of a subject's physiological history can be used for many applications, including disease diagnosis, disease monitoring, disease prognosis, drug discovery and drug evaluation. As used herein, a subject can be a mammal such as a human, non-human primate, cow, horse, pig, sheep, goat, dog, cat, or rodent. In some preferred embodiments, the subject is a human. Subjects can be free of a disease or a disorder. Subjects can be symptomatic or asymptomatic. In some embodiments, subjects are being screened to determine whether they have indicia of a disease. In other embodiments, subjects are patients known to have a disease and are being further diagnosed and/or monitored and/or treated. In preferred embodiments, a subject is a patient diagnosed as having diabetes or pre-diabetes. In other embodiments, a subject can be a subject at risk of developing diabetes. Risk factors for diabetes include genetic factors and overweight. In some embodiments, a subject has an HbA1c of greater than 5.5, preferably greater than 6.5, and more preferably greater than 7.0. In other embodiments, a subject has a blood glucose level of greater than 100 mg/dL, and more preferably greater than 120 mg/dL.

a) Disease Diagnosis

Methods of the invention are particularly useful for diagnosing diabetes and identifying the time point at which a diabetic event occurred. Methods of the invention are also useful for distinguishing between Type 1 and Type 2 diabetes. The most common forms of diabetes are Type 1 or Insulin-dependent diabetes, and Type 2 or Non-insulin-dependent diabetes, and gestational diabetes. What all forms of diabetes have in common is elevation in blood glucose or hyperglycemia. There are about 16 million diabetics in the US, with about 5-10% being Type 1 and the rest being Type 2. Type 1 diabetes is caused by an absolute insulin deficiency, and usually occurs before the age of 30, although it can occur at any age. Consequently, it was also referred to as juvenile diabetes. It is not associated with obesity and is commonly complicated by ketoacidosis. Ketoacidosis is an acute complication of diabetes, and may present as a medical emergency because of dehydration and acidosis (low blood pH). Type 2 diabetes usually develops after the age of 30 and is not associated with total loss of the ability to secrete insulin. Consequently, it was referred to as maturity-onset diabetes. Plasma insulin levels are often normal or elevated. Almost all the patients are obese, and their glucose tolerance may be restored to normal if they lose weight. They have a reduced number of active insulin receptors, and the number of these receptors can increase with weight loss. Ketoacidosis is a rare complication for Type 2 diabetic patients.

In alternative embodiments of the invention, other diseases can be diagnosed or classified. For example, the invention can be used to identify deficiencies in red blood cell production in a patient. As discussed earlier, changes in the rate of red blood cell production can be detected as shifts in the expected spacing between days as identified by blood glucose levels associated with meals.

The invention is also useful in the diagnosis, prognosis, and treatment of other diseases and disorders. Examples of diseases and disorders where this invention may be used include but are not limited to: infectious diseases, hematologic, immunologic, cardiovascular, endocrine, respiratory, rheumatologic, renal, urinary, neuronal, muscular, respiratory, gastrointestinal and neoplastic disorders. Infectious diseases include viral, bacterial, fungal, algal, parasitic, protozoal, and helminthic infections. Examples of bacterial infections include but are not limited to: staphylococcal, pneumococcal, streptococcal, meningococcal, gonococcal, pseudomonal, salmonella, hemophilus, legionella, heliobacter, shigella, cholera, brucella, tularemia, tuberculosis, syphillis, mycoplasma, chlamydia and ricketsia.

Examples of viral infections include but are not limited to: HIV, herpes, varicella, cytomegalovirus, smallpox, parvovirus, papilloma, retroviruses, enterovirus, adenoviruses, measles, mumps, rubella and rabies.

Examples of hematological disorders include but are not limited to: anemias, hemoglobinopathies, leukemias and polycythemia.

Examples of immunological disorders include but are not limited to: transplant rejection, auto-immune disorders, primary immune deficiencies, HIV, AIDS, AIDS-related disorders, allergies, anaphylaxis, vasculitis, Sjörgen's syndrome, Behcet syndrome, sarcoidoses and amyloidosis.

Examples of cardiovascular disorders where the invention can be used include but are not limited to: rheumatic fever, myocardial infarction, ischemic heart disease, heart failure, myocarditis, cardiomyopathies, atherosclerosis, arteriosclerosis, pericardial disorders, vasculitis and vascular disorders.

Examples of endocrine disorders include but are not limited to: disorders of the thyroid, parathyroid, pituitary, hypothalamus, adrenal, pancreas, ovary, testes, female reproductive tract, breast, and sexual differentiation. Other endocrine disorders include pheochromocytoma, osteoporosis, hypercalcemia, hypocalcemia, Paget's disease, bone mineral metabolism disorder, bone dysplasias, lipoprotein disorders, porphyrias, Wilson's disease, liposomal storage disease, glycogen storage disease, lipodystrophries, disorders of adipose tissue, connective tissue, amino acid metabolism disorders, disorders of purine and pyrimidine metabolism, hirsuitism, erectile dysfunction, virilization and infertility.

Examples of rheumatologic disorders include but are not limited to: arthritis, systemic lupus erythematosis, rheumatoid arthritis, scleroderma, ankylosing spondilitis, osteoarthritis, gout, psoriasis, polychrondritis and periarticular diseases.

Examples of renal disorders include but are not limited to: acute renal failure, chronic renal failure, glomerulopathies, tubular disorders, nephrolithiasis, urinary tract infections and pyelonephritis.

Examples of neuronal disorders include but are not limited to: Alzheimer's disease, Parkinson's disease, extrapyramidal disorders, ataxic disorders, autonomic disorders, cranial nerve disorders, spinal cord disorders, multiple sclerosis, demyelinating disorders, encephalitis, viral and bacterial meningitis, chronic meningitis, peripheral neuropathies, mental disorders, addiction, alcoholism, chronic fatigue syndrome and prion disorders.

Examples of muscular disorders include but are not limited to: myopathies, Guillain-Barré syndrome, immune-mediated neuropathies, Charcot-Marie-Tooth disease, inherited neuropathies, myasthemia gravis, neuromuscular junction disorders, polymyositis, dermatomyositis, and inclusion body myositis.

Examples of respiratory disorders include but are not limited to: asthma, pneumonitis, pulmonary infections, bronchitis, cystic fibrosis, interstitial lung disease, pulmonary hypertension, pulmonary thromboembolism and acute respiratory distress syndrome.

Examples of gastrointestinal disorders include but are not limited to: esophageal disorders, peptic ulcer, inflammatory bowel disease, irritable bowel syndrome, appendicitis, diverticulitis, Crohn's disease, and disorders of absorption.

Examples of neoplastic disorders include but are not limited to: melanomas, skin cancers, lung cancers, breast cancers, gastrointestinal tract cancers, liver cancers, biliary tract cancers, pancreatic cancers, urinary bladder cancers, renal cancers, prostrate cancers, metastatic cancer, lymphomas and sarcomas.

b) Disease Monitoring

Methods of the invention are particularly useful for monitoring a diabetic patient. By analyzing a single sample, an approximately 120 day glucose history can be obtained for a patient. The resolution of the analysis can be set to observe glucose changes associated with food intake. This allows a physician to obtain an accurate picture of the patient's control of their blood glucose level.

Currently the most common method used by diabetic patients for monitoring blood glucose, is to acquire a small sample of blood by sticking the finger with a lancet, and squeezing a droplet of blood onto a paper strip which is then placed on a detection device. The glucose results assist the patients in planning meals and physical activities, and also assist the doctors in optimizing the patients' insulin dosage. Unfortunately, many diabetic patients are not compliant in measuring their blood glucose regularly, and regulating their diet and physical activities, but yet their glucose levels may be at acceptable levels during their visit to the doctor's office. To get around this problem in detecting non-compliance, doctors monitor their patients' HbA1c levels every 2 to 4 months.

HbA1c is one specific type of glycated Hb, constituting approximately 80% of all glycated Hb and is formed by the spontaneous reaction of glucose with the N-terminal amino group of the Hb A beta chain. The HbA1c and the glycated Hb values have a high degree of correlation, and either may be used in the management of diabetes. As a matter of fact, some in vitro diagnostic systems measure glycated Hb but report HbA1c results. Formation of HbA1c irreversible, and the blood level depends on both the life span of the red blood cells (average 120 days for males) and the blood glucose concentration. Therefore HbA1c represents a highly-approximate blood glucose estimate that is weighted towards the several weeks prior to the assay, and does not reflect the wide fluctuations observed in actual blood glucose values. Studies have shown that quality of life improves with decreasing levels of HbA1c, and measurements every 2 to 4 months are recommended.

The gold standard for measuring HbA1c uses high performance liquid chromatography (HPLC). Other methods use affinity chromatography, ion-exchange chromatography and immunoinhibition turbidimetric techniques. In all the available methods, the first step is the production of a hemolysate by lysing the red blood cells with a special reagent. Since no near-patient testing for HbA1c is currently available, diabetic patients have to visit their doctor a second time to discuss their HbA1c results. The inconvenience to patients and the extra cost for a follow-up visit to the doctor, prompted manufacturers to develop a kit, which enables the patient to place their blood on a specially-treated test strip, which is then sent to a laboratory in a prepaid mailer. Within 1 to 2 weeks, both patients and their doctors receive the HbA1c results. By mailing in a blood sample ahead of time, the follow-up visit to the doctor can be eliminated.

The invention provides methods for simplifying the monitoring process. A patient can visit the doctor every 3-4 months to obtain an analysis of the invention. The data the doctor retrieves from the analysis is much more informative than data available from current assays. The subject's historical profile can help the doctor identify and eliminate minor deficiencies in glucose regulation by a patient.

In alternative embodiments of the invention, other diseases can be monitored. For example, the invention can be used to monitor deficiencies in red blood cell production in a patient. In some embodiments, the invention can be used to monitor anemic patients and/or cancer patients who have a low red blood cell count due to chemotherapy.

In other embodiments, the invention can be used to monitor a subject's exposure to toxins (e.g. environmental toxins or pollution). In particular the invention can be used to monitor a subject's exposure to smoke (e.g. tobacco smoke). In yet further embodiments, the invention can be used to monitor a subject's exposure to agents that affect red blood cell physiology (e.g. rate of red blood cell generation or lifespan). Examples of such agents include doping agents.

c) Therapeutic Recommendations

The invention provides significantly more information about a patient's blood glucose level than is available from a standard HbA1c assay. This allows a doctor to observe daily fluctuations in a patient's blood glucose level over a period of several months. The information obtained from this analysis can be invaluable for the doctor to fine tune a patient's therapy. The doctor may recommend a different medication or a different dose, suggest a life-style change to the patient, and/or emphasize to the patient the importance of carefully monitoring and controlling their blood glucose levels on a daily basis.

Anti-diabetic drugs have different mechanisms of action and different anti-diabetic drugs produce different effects on the blood glucose profile at different times following administration. Some anti-diabetic drugs preferentially lower the fasting blood glucose. Others preferentially lower post-prandial glucose levels. Some antidiabetic drugs reduce insulin resistance. Others stimulate insulin secretion from the pancreas. Accordingly, a doctor may base or change a treatment regimen based on the information obtained from the analysis of the blood glucose profile obtained from this invention. Treatment regimens may include one or more anti-diabetic drugs and different combinations thereof.

Anti-diabetic drugs include but are not limited to: biguanides, glucosidase inhibitors, meglitinides, sulfonylureas, thiazolidinediones and insulins. Insulins may be rapid action insulins, intermediate acting insulins, long acting insulins or a combination thereof.

It is very important for diabetic patients to control their blood glucose levels carefully to avoid complications associated with the disease. Some of the late complications of all forms of diabetes are kidney failure (nephropathy), blindness (due to retinopathy), sensory deficits (due to neuropathy). Recent long-term clinical evaluations report that failure of a patient to maintain glucose levels as close to normal as possible can contribute to these significant complications of diabetes. To adequately control the glucose levels in their blood, Type I diabetic patients must inject themselves with insulin several times daily, and must monitor their blood glucose levels between 1 and 4 times daily.

As with diabetes, the invention can be used to optimize therapies for other diseases. For example, the invention can be used to optimize medication types or doses or other treatment options for anemic patients or cancer patients.

d) Drug Discovery

Methods of the invention can be used to identify a new or improved drug from a pool of candidate drugs. Methods of the invention can also be used to identify useful doses of combinations of drugs. According to the invention, a candidate drug can be administered to a subject for a period of time. The effect of the drug on a specific physiological activity of the subject can be evaluated over the period of time from a single sample analysis as described herein. For example, new drugs or new dosages or combinations of existing drugs for diabetes treatment can be identified by monitoring a subject's blood glucose level fluctuations in response to the candidate drug, dose, or combination.

In one aspect of the invention, different general classes of disease may be identified using the high resolution analytical methods of the invention. For example, methods of the invention may provide sufficient information of daily blood glucose fluctuations to identify new sub-classes of diabetic individuals with similar traits that were not identifiable using current blood glucose monitoring methods. Accordingly, new drugs, drug doses, or drug combinations may be optimized for these sub-classes of patients. In addition, new drugs, drug doses, or drug combinations may be optimized for individual patients.

e) Drug Evaluation

Similarly, new or existing drugs, drug doses, or drug combinations can be evaluated for unwanted side-effects by evaluating a subject's physiological profile after the subject has received the new drug, drug dose, or drug combination for a period of time. As used herein a drug includes a natural or synthetic product or a pharmaceutical agent.

Applications and Devices

The invention also provides devices that are useful for separating and sorting cells as a function of their amount of cellular modification. The following chromatography and electrophoresis devices are useful for separating red blood cells with different levels of glycation. However, they can be adapted for separating other cells as a function of different forms of cellular modification.

a) Chromatography Devices

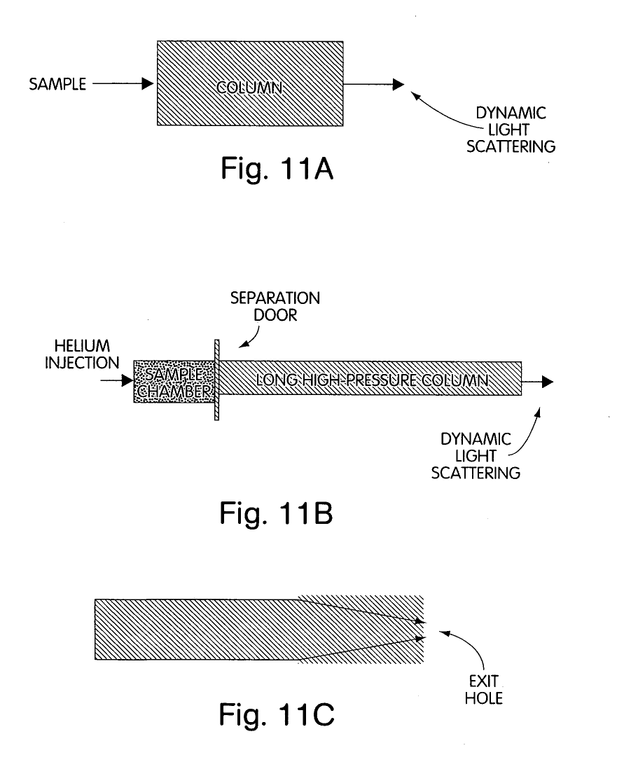

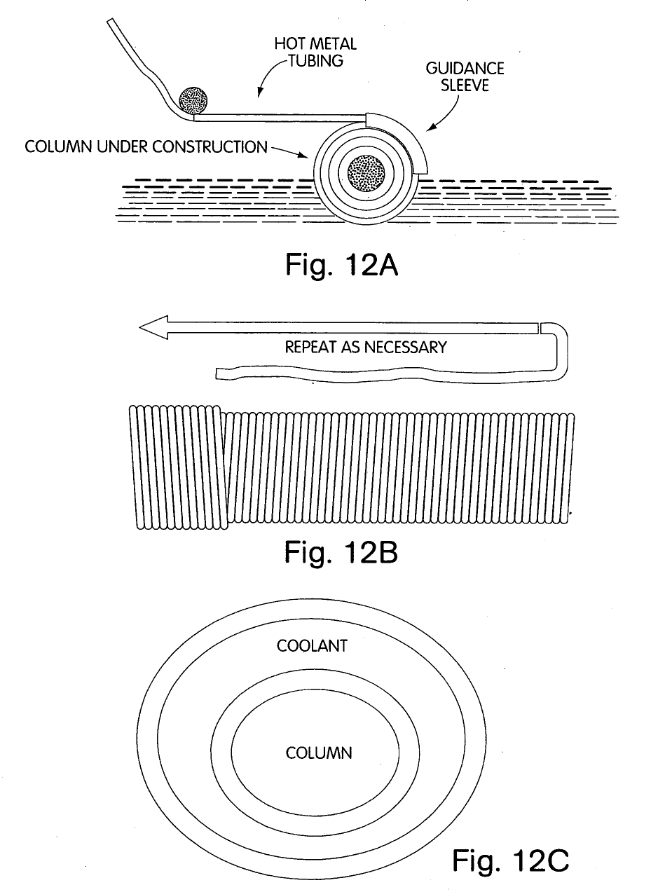

In one aspect of the invention, cellular modification amounts can be measured using non-destructive chromatography and a measuring device. A preferred measuring device includes light scattering equipment (for example, from Wyatt Technology, Santa Barbara, Calif.). These devices can be used to obtain raw data representing cells distributed as a function of their degree of cellular modification. The data from these devices can be sorted as it is being captured. Alternatively, the data can be sorted after it is captured. FIGS. 11 and 12 show an embodiment of a chromatography device of the invention and an example of a method for manufacturing the device.

In one embodiment, a blood sample can be processed as follows. The blood sample is spun in a centrifuge. After obtaining the crit and discarding the plasma, the cells are placed in a container. In preferred embodiments, the cells are treated so that they do not stick together. A small amount of distilled water, isotonic solution or other liquid that is unlikely to bind the hemoglobin can be added. As little water as possible is added, while still facilitating the molecular reaction with the next additive. Molecules of a “very-large-molecular-volume-molecule” are added to bind to the hemoglobin. These molecules are preferably much larger than glucose. A high pressure gas chromatography is then applied to the sample as follows:

A sample first is placed in the sample chamber. The column is then pressurized with an inert gas to a high pressure. A preferred gas is helium. However, other gases may be used. The separation door is opened slowly. However, the gas (e.g. helium) is not yet injected. After waiting long enough for approximately all of the cells to diffuse into the high-pressure column, the exit point on the right is opened while simultaneously injecting helium from the left in order to maintain constant pressure in the column. The exit hole is preferably small so that the exit rate of the cells can be handled easily by the light scattering equipment. The shape of the right end of the column is preferably conical.

One embodiment for manufacturing a chromatography column is shown in FIG. 12. In the side view of FIG. 12A, the column is constantly being rotated clockwise as the metal tubing is applied. The curvature of the guidance sleeve is altered as the column spirals outward. In preferred embodiments, the column is volume efficient. In one embodiment, nearly 75 m/L of column length can be achieved with a 1/10 cm² column cross sectional area. In preferred embodiments, both ends of the column are at the same end of the structure. The entire structure can be encased in metal to provide additional support during the application of high pressure. This technique allows very long columns to be built so that they fit in a small volume. In some embodiments it may be necessary to use liquid cooling of the column to avoid damage to the RBCs. FIG. 12C shows an embodiment with metal tubing surrounded by an enclosed coolant volume. The coolant is pumped through the tubing's outer diameter. The concentric tubes can touch. However, the cross sectional area of the outside diameter of the inner tubing is preferably not so large that it can block the flow of the coolant in the outer tubing. Any liquid or gas coolant can be used. Examples of preferred coolants include water and liquid nitrogen.

b) Electrophoresis Devices

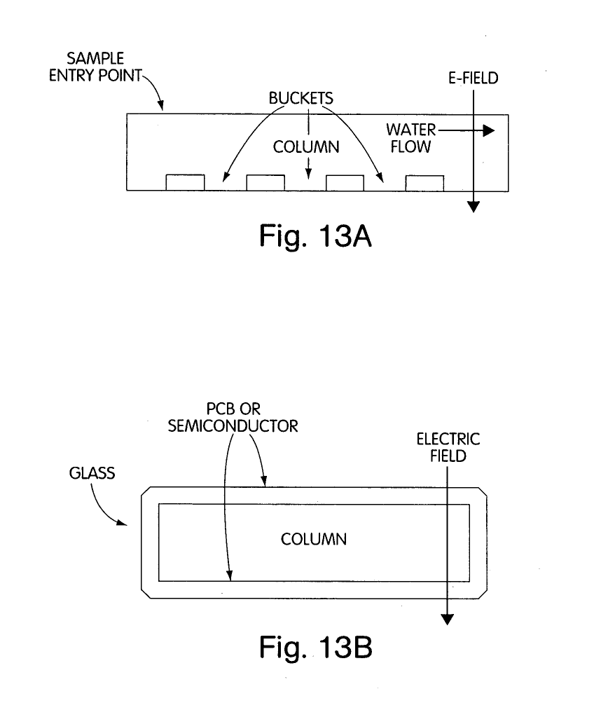

In another aspect of the invention, cellular modification amounts can be measured using an electrophoretic device. An electrophoretic device can be used to obtain raw data representing cells distributed as a function of their degree of cellular modification. The data can be sorted as it is being captured. Alternatively, the data can be sorted after it is captured. FIG. 13 shows an embodiment of an electrophoretic device of the invention.

In one embodiment, a low intensity electric field is applied to an ion exposed sample traveling in a slow-moving liquid. In a preferred embodiment, a low-intensity dc electric field is used to precipitate cells out of the sample as it travels along a volume or channel with a slow-moving liquid or gel. As in the previous method, the sample is prepared by spinning a blood sample in a centrifuge. After obtaining the crit and discarding the plasma, cells are placed in a container with a small amount of liquid, preferably distilled water, isotonic solution, or some other liquid that unlikely to bind with the hemoglobin. Preferably, the cells don't stick together. Preferably, the molecular reaction with the next additive is facilitated. In one embodiment, the sample is exposed to one or more ions that bind to the hemoglobin. Preferred ions are either ions that interact specifically with glycated hemoglobin or ions that interact specifically with non-glycated hemoblobin. Preferred ions include, for example, ionized glucose phosphates and other ions that bind specifically to either glycated or non-glycated hemoglobin. In one embodiment, an ion that binds to unglycated Hb results in all of the cells carrying an electrical charge, those originally having the least amount of glucose bound, the youngest cells, being most electrically charged after exposure to the ions. In another embodiment, an ion that binds glycated Hb results in cells carrying an electrical charge if the cells contain glycated Hb, and the most electrically charged cells being those with the highest amount of glycated Hb (the oldest cells).

A sample can be introduced to an electrophoretic device by slowly injecting the sample into the top part of a column. In FIG. 13, a liquid or gel support is moving left to right at a slow rate. A weak electric field is applied in a normal orientation relative to the direction of fluid flow. This forces ions that are attached to the cells toward the bottom of the column. As each cell reaches the lower edge of the column, it gets stuck in a “bucket” shown on the lower edge. Each cell remains in a bucket until a subsequent step in the procedure. In one embodiment, cells with the smallest number of ions attached are the oldest cells, and they will travel the longest distance along the column before reaching a bucket.

After all of the cells have reached a bucket, the liquid flow is stopped. The electric field that was used to push the cells to the bottom of the column is removed. In one embodiment, a vertically-upward flow of water is created in the column using holes on or near the bottom and the top of the column. Alternatively, an electric field of opposite polarity relative to the initial field is applied. These and other methods can be used to move the cells to the top of the column. The upward movement of the cells is reduced or stopped and the left to right motion of liquid is reapplied until all of the cells have left the bucket part of the column. Older cells leave the column first. As the cells leave the bucket part of the column, many different techniques can be used to recover the cell density as a function of time. This procedure provides raw data. However, in most embodiments a plurality of cells will be captured in each bucket. Therefore, the raw cell data is already divided into ranges and the ordered output is a CDP.

Using semiconductor industry etching techniques, buckets can be created of any size. In some embodiments, the buckets may be so small that they are approximately the same size as a cell. As such, these buckets are only big enough to receive a single cell. In other embodiments, the width of the bucket in the direction normal to the flow allows for multiple cells to be deposited in a bucket. In other embodiments, the holes on top and bottom of the column can be much smaller than a cell so that none of the sample is lost in the vertical flow step. Alternatively, printed circuit board (PCB) etching can be used to make a less expensive version of the electrophoretic device. In addition, the holes near the top and bottom can be placed on the sides of the column.

In some embodiments, the device can be a PCB with 50-100 layers, that uses buried via technology to implement the column. The number and thickness of the layers determines the height of the column and thus the vertical distance cells travel while they're being separated or differentiated. In one embodiment, the column is the planar width of a panel, about four feet, and 5 mils long buckets are constructed with 5 mils between buckets. However, longer or shorter buckets can be used with longer or shorter distances between buckets. The sizes of the different buckets can be different. The sizes of the buckets and the distances between buckets can be different. The embodiment described above will have about five thousand buckets. These buckets can be used to represent about a 30 minute amount of cells in each bucket assuming that the cells in the sample are evenly distributed across the buckets. There are 120*24=2880 hours represented in a blood glucose analysis of the invention (for a male subject with an RBC life of 120 days). In some embodiments, manufactured PCBs will include more buckets. As discussed herein, there are preferably more buckets than times returned in the analysis, because an increased number of buckets provides higher resolution and produces more accurate results for each time returned. In addition, the properties of cells from different subjects will be different. As a result, cells from different subjects may be distributed over different ranges of buckets. Accordingly, a preferred device may have a series of buckets covering a large range. In preferred embodiments, a subject's blood sample will be distributed over only 10%-50% of the buckets in such a device.

Holes used to control the flow of the medium can either be drilled or designed into a PCB. A metal plate can cover the top of the column. However, in preferred embodiments, the “plate” for the other electrode is a metal layer that is present solely at the bottoms of the buckets and is connected just beyond the left and right edges of the column (under the “glass” in FIG. 13). Preferably, there is no electrode on or under the surface area between the buckets.

c) Computer Systems

Various embodiments according to the invention may be implemented on one or more computer systems. These computer systems, may be, for example, general-purpose computers such as those based on Intel PENTIUM-type processor, Motorola PowerPC, Sun UltraSPARC, Hewlett-Packard PA-RISC processors, or any other type of processor. It should be appreciated that one or more of any type computer system may be used according to various embodiments of the invention. Further, the software design system may be located on a single computer or may be distributed among a plurality of computers attached by a communications network.

A general-purpose computer system according to one embodiment of the invention is configured to perform methods of the invention. It should be appreciated that the system may perform other functions, including displaying the data, and the invention is not limited to having any particular function or set of functions.

For example, various aspects of the invention may be implemented as specialized software executing in a general-purpose computer system. The computer system may include a processor connected to one or more memory devices, such as a disk drive, memory, or other device for storing data. Memory is typically used for storing programs and data during operation of the computer system. Components of the computer system may be coupled by an interconnection mechanism, which may include one or more busses (e.g., between components that are integrated within a same machine) and/or a network (e.g., between components that reside on separate discrete machines). The interconnection mechanism enables communications (e.g., data, instructions) to be exchanged between system components of the system. The computer system also includes one or more input devices, for example, a keyboard, mouse, trackball, microphone, touch screen, and one or more output devices, for example, a printing device, display screen, speaker. In addition, the computer system may contain one or more interfaces that connect the computer system to a communication network (in addition or as an alternative to the interconnection mechanism).

The storage system typically includes a computer readable and writeable nonvolatile recording medium in which signals are stored that define a program to be executed by the processor or information stored on or in the medium to be processed by the program. The medium may, for example, be a disk or flash memory. Typically, in operation, the processor causes data to be read from the nonvolatile recording medium into another memory that allows for faster access to the information by the processor than does the recording medium. This memory is typically a volatile, random access memory such as a dynamic random access memory (DRAM) or static memory (SRAM). It may be located in the storage system or in the memory system. The processor generally manipulates the data within the integrated circuit memory and then copies the data to the medium after processing is completed. A variety of mechanisms are known for managing data movement between the medium and the integrated circuit memory element and the invention is not limited thereto. The invention is not limited to a particular memory system or storage system.

The computer system may include specially-programmed, special-purpose hardware, for example, an application-specific integrated circuit (ASIC). Aspects of the invention may be implemented in software, hardware or firmware, or any combination thereof. Further, such methods, acts, systems, system elements and components thereof may be implemented as part of the computer system described above or as an independent component.

Various aspects of the invention may be practiced on one or more computers having different architectures or components.

A computer system may be a general-purpose computer system that is programmable using a high-level computer programming language. The computer system may be also implemented using specially programmed, special purpose hardware. In one embodiment of a computer system, the processor is typically a commercially available processor such as the well-known Pentium class processor available from the Intel Corporation. Many other processors are available. Such a processor usually executes an operating system which may be, for example, the Windows 95, Windows 98, Windows NT, Windows 2000 (Windows ME) or Windows XP operating systems available from the Microsoft Corporation, MAC OS System X available from Apple Computer, the Solaris Operating System available from Sun Microsystems, or UNIX available from various sources. Many other operating systems may be used.

The processor and operating system together define a computer platform for which application programs in high-level programming languages are written. It should be understood that the invention is not limited to a particular computer system platform, processor, operating system, or network. Also, it should be apparent to those skilled in the art that the present invention is not limited to a specific programming language or computer system. Further, it should be appreciated that other appropriate programming languages and other appropriate computer systems could also be used.

One or more portions of the computer system may be distributed across one or more computer systems (not shown) coupled to a communications network. These computer systems also may be general-purpose computer systems. For example, various aspects of the invention may be distributed among one or more computer systems configured to provide a service (e.g., servers) to one or more client computers, or to perform an overall task as part of a distributed system. For example, various aspects of the invention may be performed on a client-server system that includes components distributed among one or more server systems that perform various functions according to various embodiments of the invention. These components may be executable, intermediate (e.g., IL) or interpreted (e.g., Java) code which communicate over a communication network (e.g., the Internet) using a communication protocol (e.g., TCP/IP).

It should be appreciated that the invention is not limited to executing on any particular system or group of systems. Also, it should be appreciated that the invention is not limited to any particular distributed architecture, network, or communication protocol.

Various embodiments of the present invention may be programmed using an object-oriented programming language, such as SmallTalk, Java, C++, Ada, or C# (C-Sharp). Other object-oriented programming languages may also be used. Alternatively, functional, scripting, and/or logical programming languages may be used. Various aspects of the invention may be implemented in a non-programmed environment (e.g., documents created in HTML, XML or other format that, when viewed in a window of a browser program, render aspects of a graphical-user interface (GUI) or perform other functions). Various aspects of the invention may be implemented as programmed or non-programmed elements, or any combination thereof.

Data Structures

As used herein, a “data structure” is an arrangement of data defined by computer-readable signals. These signals may be read by a computer system, stored on a medium associated with a computer system (e.g., in a memory, on a disk, etc.) and may be transmitted to one or more other computer systems over a communications medium such as, for example, a network.

Networks

As used herein, a “network” or a “communications network” is group of two or more devices interconnected by one or more segments of transmission media on which communications may be exchanged between the devices. Each segment may be any of a plurality of types of transmission media, including one or more electrical or optical wires or cables made of metal and/or optical fiber, air (e.g., using wireless transmission over carrier waves) or any combination of these transmission media.

The function and advantage of these and other embodiments of the present invention will be more fully understood from the examples described below. The following examples are intended to illustrate the benefits of the present invention, but do not exemplify the full scope of the invention.

### EXAMPLES

### Example 1

### Blood Processing Methods

In one embodiment, a blood sample is obtained from a subject and is preferably mixed with a preservative or a solution (e.g, isotonic solution) to keep the red blood cells intact. Preservatives and solutions that keep cells intact are well known in the art.

### Example 2

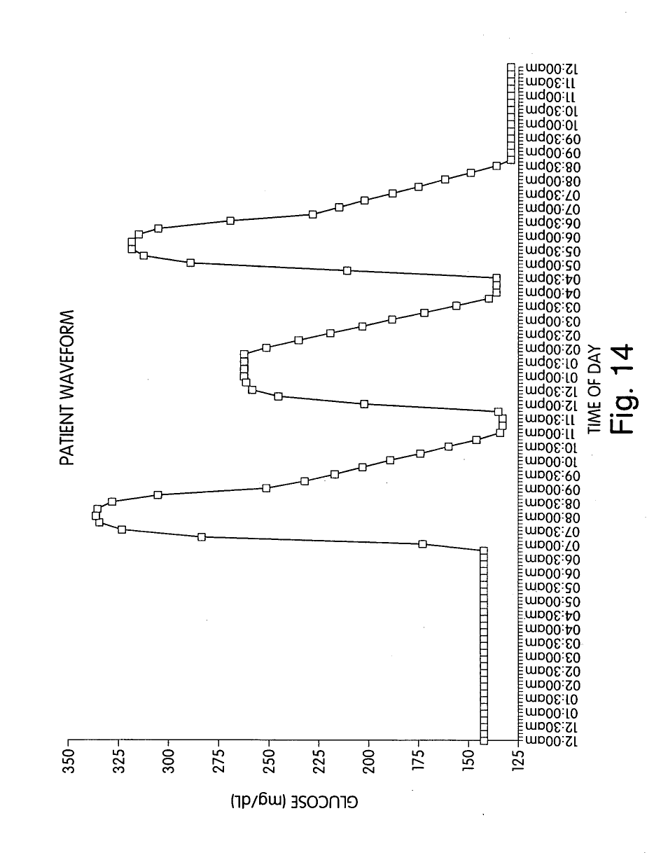

A computer algorithm can be used to calculate a blood glucose profile from a CDP. A model of actual daily glucose fluctuations for a diabetic patient was generated using a computer algorithm. These fluctuations are shown in FIG. 14. The recovered blood glucose history is provided for a day a little more than one week before the sample was taken (day 112 out of a 120 day history) in the computer-generated model. The data provides an accurate 1 data point/15 min. profile showing that the information is in fact contained in the glycation of the cell sample in this model. A rough inspection of this data shows that it is accurate to within 1% of the initial input data. In this case, because of the extremely small cell sample, more accurate information can be obtained by bypassing a CDP analysis and looking at individual cells. No statistical variance or variance in RBC production was modeled in this simulation.

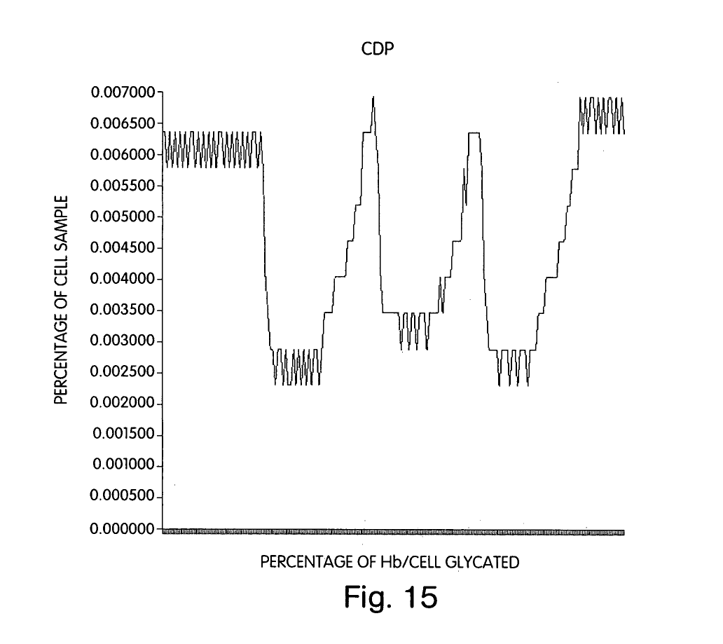

The CDP shown in FIG. 15 relates to day 112 (the day illustrated in FIG. 14). This CDP was generated by dividing the data for day 112 into 180 ranges of equal width.

Table 1 outlines the part of the program that illustrates an embodiment of the method for retrieving an hourly blood glucose level from the CDP of FIG. 15.

TABLE 1

| Step | Program |
|---|---|
| 1. Set up the parameters | cdp_sum=0.0; for |
| to obtain a history from a | (range=0;range<180;range++){cdp_sum=cdp_sum+cdp_{—} |
| CDP. | y[range];} fprintf(OUTPUT,“sum=%9.6f\n”,cdp_sum); |
| 2. Break the day's cells | cdp_halfbin_size=cdp_sum/48.0; |
| into 48 groups, one per | |
| half hour. | |
| 3. First “target” break | cdp_target=cdp_halfbin_size; |
| point is at 12:30 am. | |
| 4. Initialize | cdp_total=0; range=0; halfpast=0; |
| total/range/halfpast“bin”c | |
| enter range. | |
| 5. Keep adding a | while ((halfpast<24)&&(range<180)){while |
| percentage of cells until | ((cdp_total<cdp_target)&&(range<180)){ |
| reaching the next “target.” | cdp_total=cdp_total+cdp_y[range]; range++;} |
| | cdp_halfhour_ptr[halfpast]=range; halfpast++; |
| 6. Make a new target for | cdp_target=cdp_target+(2*cdp_halfbin_size); } |
| one hour later. | |
| 7. Print out glycation of | for (bin=0;bin<24;bin++) fprintf(OUTPUT,“bin%2d: |
| each “hour plus thirty | cdp_x[bin]=%9.6f\n”, bin,cdp_x[cdp_halfhour_ptr[bin]]); |
| minutes” point. | |
| 8. Calculate “delta | for (halfpast=0;halfpast<24;halfpast++) |
| cdp_x”: the glycation of | if(halfpast!=0) |
| left edge of the bin minus | fprintf(OUTPUT,“hour %2d%s: delta |
| the glycation of the right | cdp_x=%9.6f glucose %dmg/dL\n”, |
| edge of bin; | halfpast<13?halfpast:halfpast- |
| multiply by | 12,halfpast<12?“am”:“pm”, |
| “GLYMULT” to convert | (cdp_x[cdp_halfhour_ptr[halfpast-1]]) |
| to mg/dL. | -(cdp_x[cdp_halfhour_ptr[halfpast-0]]), |
| | (int)(GLYMULT*((cdp_x[cdp_halfhour_ptr[halfpast-1]]) |
| | (cdp_x[cdp_halfhour_ptr[halfpast-0]])))); |

In step 1, the parameters for analyzing the CDP are provided. The CDP for day 112 is being analyzed. The CDP contains 180 equal steps, each CDP step representing the glycation difference between the beginning and end of the day divided by 180. The sum=0.833333 printout is an optional step that controls for correctness. In this example, it represents 100/120 of the total sample. The value of the CDP summed over the 120 days can be compared to the expected value based on an HbA1c measurement.

In step 2, the day's cells are divided into 48 half-bins. Each bin corresponds to 1 hour of time for the day.

In step 3, the first target point for the analysis is set at 12.30 am.

In step 4, the analysis is initialized.

In step 5, ranges are added until the next target is reached (until a full bin's worth of cells has been added).

In step 6, a new target is set 1 hour later.

In step 7, the glycation for each hour plus thirty minutes is printed. This is an optional steps. The results are shown in Table 2.

In step 8, the glycation differences (delta cdp_x) for each bin are calculated by subtracting the right edge from the left edge of each bin. The glycation differences are then converted into glucose levels.

In further embodiments, an additional step (step 9) is included to correct for sources of non-linearity such as those described herein.

The results are shown in Table 3.

TABLE 2

| bin # | cdp-x[bin] |
|---|---|
| 0 | 1.243861 |
| 1 | 1.238764 |
| 2 | 1.233666 |
| 3 | 1.229418 |
| 4 | 1.224321 |
| 5 | 1.219223 |
| 6 | 1.214975 |
| 7 | 1.207329 |
| 8 | 1.196284 |
| 9 | 1.186938 |
| 10 | 1.180991 |
| 11 | 1.175893 |
| 12 | 1.169097 |
| 13 | 1.160601 |
| 14 | 1.152105 |
| 15 | 1.145308 |
| 16 | 1.141060 |
| 17 | 1.132564 |
| 18 | 1.121519 |
| 19 | 1.113873 |
| 20 | 1.108775 |
| 21 | 1.104527 |
| 22 | 1.099429 |
| 23 | 1.095181 |

TABLE 3

| Hour | delta cdp_x | glucose (mg/dL) |
|---|---|---|
| 1 am | 0.005098 | 149 |
| 2 am | 0.005098 | 149 |
| 3 am | 0.004248 | 124 |
| 4 am | 0.005098 | 149 |
| 5 am | 0.005098 | 149 |
| 6 am | 0.004248 | 124 |
| 7 am | 0.007646 | 224 |
| 8 am | 0.011045 | 324 |
| 9 am | 0.009346 | 274 |
| 10 am | 0.005947 | 174 |
| 11 am | 0.005098 | 149 |
| 12 pm | 0.006797 | 199 |
| 1 pm | 0.008496 | 249 |
| 2 pm | 0.008496 | 249 |
| 3 pm | 0.006797 | 199 |
| 4 pm | 0.004248 | 124 |
| 5 pm | 0.008496 | 249 |
| 6 pm | 0.011045 | 324 |
| 7 pm | 0.007646 | 224 |
| 8 pm | 0.005098 | 149 |
| 9 pm | 0.004248 | 124 |
| 10 pm | 0.005098 | 149 |
| 11 pm | 0.004248 | 124 |

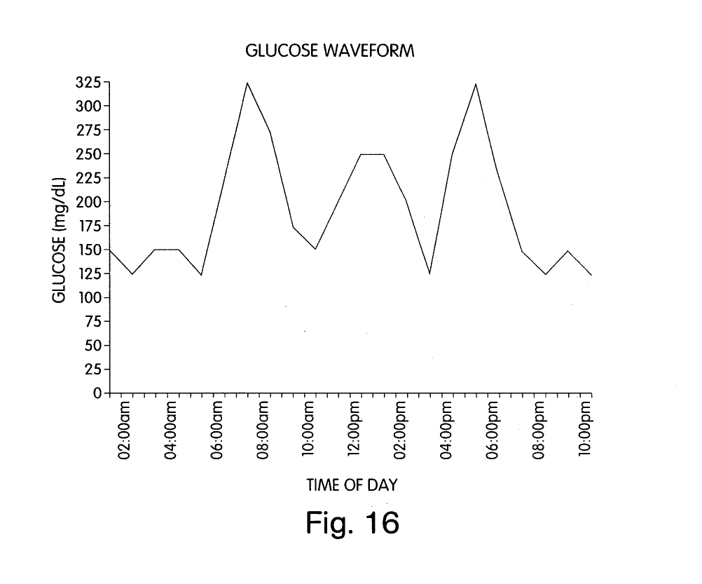

FIG. 16 shows the hour by hour glucose history obtained form the analysis of the CDP section discussed above. The patient history shown in FIG. 16 shows that patient glucose level responses to three meals can be recovered from the CDP. This illustrates the type and quality of information that can be retrieved from a CDP or from ordered cellular modification data. The profile shown in FIG. 16 also illustrates a prolonged time at high glucose and a slow recovery in response to elevated glucose after the second meal of the day. This slow recovery suggests that the patient's long-term medication, but not short-term, is controlling the patient's blood glucose level at this stage of the day. This information is very useful for patient diagnostic, monitoring, or therapy. A doctor could identify this slow recovery and determine whether it is due to inappropriate medication or dosage or to lack of patient compliance with a prescribed treatment.

The analysis discussed above illustrates an embodiment of the invention where sorted cellular measurements are obtained, the resulting total glycation width is divided into smaller ranges, the ranges are grouped into bins of a preselected width corresponding to a time period of interest (e.g. a minute, an hour, a day, a week, or a month), and the glycation change for each bin is obtained by calculating the difference between the leftmost and the rightmost edges of the bin. These edges are represented, respectively, by the leftmost and rightmost ranges in that bin. The glycation differences can then be converted to blood glucose levels. Generally, the resulting blood glucose level for each bin is assigned to a time point in the center of the time represented by the bin. However, the resulting blood glucose level for a bin could be assigned to any time point associated with that bin without any significant loss of information.

Having now described some illustrative embodiments of the invention, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the invention. In particular, although many of the examples presented herein involve specific combinations of method acts or system elements, it should be understood that those acts and those elements may be combined in other ways to accomplish the same objectives. Acts, elements and features discussed only in connection with one embodiment are not intended to be excluded from a similar role in other embodiments. Further, for the one or more means-plus-function limitations recited in the following claims, the means are not intended to be limited to the means disclosed herein for performing the recited function, but are intended to cover in scope any means, known now or later developed, for performing the recited function. Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

All references, patents and patent publications that are recited in this application are incorporated in their entirety herein by reference.

1. A method of obtaining a subject's physiological history from a sample analysis, the method comprising the steps of:
(a) measuring an amount of a cellular modification for each of a plurality of cells from a biological sample;
(b) sorting the cell measurements from step (a) as a function of the amount of modification; and,
(c) determining a physiological history for the subject by analyzing the ordered cell measurements.

2. The method of claim 1, wherein the physiological history is indicative of a disease or disorder in the subject.

3. The method of claim 2, wherein the disease or disorder is diabetes.

4. The method of claim 3, wherein the diabetes is Type I diabetes.

5. The method of claim 3, wherein the diabetes is Type II diabetes.

6-18. (canceled)

19. A method of obtaining a cell distribution profile of a patient, the method comprising the steps of:
(a) obtaining a biological sample comprising intact cells; and
(b) determining a cell distribution profile for a plurality of the intact cells using a procedure that sorts the intact cells based on an amount of a physiological variable in each of the sorted cells.

20-24. (canceled)

25. A method for recovering the blood glucose level history of a patient diagnosed with diabetes, the method comprising the steps of:
(a) obtaining a blood sample comprising intact cells
(b) obtaining a cell distribution profile by sorting a plurality of the intact cells based on the amount of glycated hemoglobin in each cell; and,
(c) determining the patient's glucose level history based on the cell distribution profile.

26. The method of claim 25, wherein the patient's blood glucose level history is obtained by comparing the patient's cell distribution profile to one or more reference profiles.

27. The method of claim 25, wherein the patient's blood glucose level history is obtained using an algorithm.

28. The method of claim 27, wherein the algorithm corrects for non-linearity in the rate of hemoglobin glycation.

29. The method of claim 27, wherein the algorithm uses patient specific input data.

30. The method of claim 29, wherein the patient specific input data is selected from the group consisting of the patient's age, gender, and weight.

31. The method of claim 27, wherein the algorithm uses a value for the average life span of a red blood cell.

32. The method of claim 25, wherein the intact cells are sorted using a procedure selected from the group consisting of chromatography, electrophoresis, mass spectrometry, and cell sorting.

33. The method of claim 25, comprising the additional step of determining whether the patient has Type I, Type II, or gestational diabetes.

34. The method of claim 25, comprising the additional step of determining a treatment regimen for the patient.

35. The method of claim 25, comprising the step of obtaining a second blood sample at a second time and performing steps (b) and (c) on the second blood sample.

36. The method of claim 25, wherein the glycosylated hemoglobin is labeled.

37. A chromatography device for separating blood cells based on levels of glycosylated hemoglobin, the device comprising:
(a) a high pressure column comprising a sample chamber and a tapered exit hole;
(b) a dynamic light scattering detector connected to the exit hole; and,
(c) a gas powered injector connected to the sample chamber.

38. An electrophoretic device for separating blood cells based on levels of glycosylated hemoglobin, the device comprising:
(a) a liquid flow column with a sample entry point and a plurality of collection points along the length of the column; and,
(b) an electromagnetic field generator associated with the column, wherein the electromagnetic field is normal to the direction of the liquid flow.
